(19) United States
(12) Patent Application Publication
(10) Pub. No.: US 2002/0115082 A1
(43) Pub. Date: Aug. 22, 2002

(54) Methods and compositions for polynucleotide analysis using generic molecular beacons

(76) Inventors: Vince Phillips, Sunnyvale, CA (US); Andrew R. Watson, Belmont, CA (US); Edith Wong, Danville, CA (US)

Correspondence Address: ROBINS & PASTERNAK LLP, Suite 200, 90 Middlefield Road, Menlo Park, CA, 94025, US

(21) Appl. No.: 09/945,379

(22) Filed: Aug. 31, 2001

Related U.S. Application Data
(60) Provisional application No. 60/230,186, filed on Sep. 1, 2000.

(51) Int. Cl. C12Q 1/68; C12P 19/34

(52) U.S. Cl. 435/6; 435/91.2

(57) ABSTRACT

Methods, compositions and articles of manufacture for assaying a sample for an amplification product from a target polynucleotide are provided. An amplification reaction is used to produce the amplification product from the target polynucleotide so that it can be used to indirectly assay the sample for the target polynucleotide. A sample suspected of containing the target polynucleotide is contacted with first and second primers to amplify the target polynucleotide; the first primer comprises a tag sequence, the complement of which is formed on the opposite strand during amplification and is referred to as a capture sequence. That opposite strand is referred to as a second primer extension product or an amplification product. A probe polynucleotide is provided that is a molecular beacon and can bind to the capture sequence to form an amplification product detection complex. Methods of detecting the amplification product detection complex thus produced are also provided, as are amplification product assay arrays, along with methods of forming the same. The methods are particularly useful in multiplex settings where a plurality of target polynucleotides are to be assayed. Kits comprising reagents for performing such methods are also provided.

### CROSS-REFERENCE TO RELATED APPLICATIONS

This application is related to provisional patent application Ser. No. 60/230,186, filed Sep. 1, 2000, from which priority is claimed under 35 USC §119(e)(1) and which is incorporated herein by reference in its entirety.

### TECHNICAL FIELD

This invention relates to methods and compositions for the analysis of polynucleotides in a sample.

### BACKGROUND OF THE INVENTION

Adverse reactions to therapeutic drugs have been estimated to kill over 100,000 hospitalized patients in the U.S. each year (Lazarou et al. (1998) JAMA 279(15):1200-5). This figure does not include intentional overdoses leading to hospitalization that ultimately prove fatal. An additional 2.2 million serious nonfatal adverse drug reactions have been estimated to occur annually.

The problem of the varied responses of individual patients to particular drug therapies is well known, but little progress has been made towards anticipating patients' varied drug metabolisms prior to treatment. The standard approach in administering drugs has been to prescribe the recommended dosage for a given condition to an affected patient, in some cases adjusting for the patient's weight. If the patient does not improve, the dosage is increased or an alternative drug is tried. Conversely, if adverse side effects occur, the dosage may be lowered or an alternative drug employed.

Drugs that exhibit serious side effects may never be approved by regulatory authorities or, if approved before such side effects are identified, can be withdrawn from the market if even a small percentage of treated patients are so affected. This can occur despite the fact that such drugs may have great therapeutic benefit in the majority of patients.

For example, acute lymphocytic leukemia (ALL) affects thousands of children each year in the United States. Treatment with chemotherapeutic agents now leads to remission in over 90% of the cases. 6-mercaptopurine (6-MP) is one agent used to treat ALL. However, the normal treatment dose of 6-MP is toxic for one in 300 patients and can kill rather than cure. The 6-MP sensitivity exhibited by rare ALL patients has been linked to a deficiency in thiopurine S-methyltransferase (TPMT) activity (Krynetski et al. (1996) Pharm. Res. 16(3):342-9). Patients deficient in this enzyme can be treated with lower doses of 6-MP to achieve the same therapeutic plasma levels while avoiding adverse toxicity if the prescribing physician is aware of the metabolic deficiency. Metabolism of similar drugs such as azathiopurine and thioguanine used in the treatment of rheumatoid arthritis, leukemia and Crohn's disease is also affected in patients who are deficient in TPMT.

Cytochrome P-450 CYP2D6 (debrisoquin hydroxylase) is the primary enzyme responsible for human metabolism of fluoxetine (Prozac®), as well as codeine, amphetamines, methadone, and several antidepressants and neuroleptics. At least twenty variants of the CYP2D6 gene are now known to result in poor metabolism of Prozac® and other drugs (Wong et al. (2000) Ann. Acad. Med. Singapore 29(3):401-6). Approximately 7-10% of Caucasians are poor metabolizers of Prozac®, and reach higher than expected plasma levels when treated with a standard dosage.

Potentially fatal adverse drug reactions are now known to be associated with altered metabolism by patients harboring variants in a number of genes, including in the NAT2 gene affecting isoniazid metabolism, in the CYP2C9 gene affecting warfarin metabolism, in the DPD gene affecting 5-fluorouracil metabolism, and in the KCNE2 gene affecting clarithromycin metabolism (Grant et al. (2000) Pharmacol. 61(3):204-11; Taube et al. (2000) Blood 96(5):1816-9; Meinsma et al. (1995) DNA Cell Biol. 14(1):1-6; Sesti et al. (2000) Proc. Natl Acad. Sci. USA 97(19):10613-8).

Accordingly, there is a need in the art for methods of analyzing samples for particular polynucleotides, and for devices, compositions and articles of manufacture useful in such methods.

### SUMMARY OF THE INVENTION

Methods, compositions and articles for assaying for an “amplification product” prepared from a target polynucleotide in a sample are provided.

In one aspect, a method is provided comprising contacting a sample suspected of containing a target oligonucleotide with a first primer comprising a target complementary region and a target noncomplementary region to form a first primer extension product; contacting the first primer extension product with a second primer to form a second primer extension product (the “amplification product”), which amplification product comprises a nucleotide region complementary to the target noncomplementary region in the first primer extension product (the “capture sequence”); and contacting the amplification product with a first “probe polynucleotide” that comprises a molecular beacon having an oligonucleotide segment complementary to the capture sequence. The probe polynucleotide can be conjugated to a substrate, and may be presented in the form of an array of a plurality of probe polynucleotides specific for different amplification products. Amplification reactions can be incorporated into the methods.

The probe polynucleotide binds to a capture sequence that does not normally occur in the amplification product produced from the target polynucleotide. The capture sequence is introduced into the amplification product via primer-mediated extension from a template. A first primer (FIG. 1) is used to prime the synthesis of an oligonucleotide complementary to the target polynucleotide, forming a first primer extension product (“PEP”) (FIG. 2). The first primer comprises a target complementary region (“target CR” or “TCR”) at its 3′ end that is complementary to the target polynucleotide, and a target noncomplementary region (“target NCR” or “TNCR”) that does not normally occur adjacent to the target CR (FIG. 1). The first PEP thus comprises an extended TCR and the TNCR. A second primer, i.e., a “reverse” primer, is used to prime synthesis from the first PEP to form a second PEP, which can be prepared using a labeled nucleotide to form a labeled second PEP (FIG. 3). The second PEP is also referred to herein as the “amplification product.” The second PEP comprises a sequence that is complementary to the TNCR in the first PEP; this TNCR-complementary sequence in the second PEP is referred to herein as the “capture sequence.” The capture sequence binds to the probe polynucleotide to form an amplification product detection complex (FIG. 4). The probe polynucleotide can be associated with substrate. Identification of the amplification product detection complex demonstrates that the amplification product was formed and the target polynucleotide was present in the sample.

Binding of the capture sequence to the probe polynucleotide results in the formation of an amplification detection assay complex. Where a plurality of different probe polynucleotides are attached to the same substrate, binding of a plurality of corresponding different labeled amplification products results in the formation of an amplification product assay array.

Kits comprising reagents useful for performing the methods of the invention are also provided.

The methods are particularly useful in multiplex settings where a plurality of different probe polynucleotides are used to assay for a plurality of different target polynucleotides. The large number of distinguishable semiconductor nanocrystal labels can be employed to simultaneously analyze differently labeled probe polynucleotides.

Methods of the invention can optionally be implemented in a homogeneous format. This allows for higher assay throughput due to fewer manipulations of the sample, and decreased cross-contamination resulting in more reliable assays and less downtime from cross-contamination. If real time monitoring is used, the entire assay can be disposed of without opening a sealed assay chamber such as a sealed microplate, thus further decreasing the risk of cross-contamination.

### BRIEF DESCRIPTION OF THE DRAWINGS

FIG. 1 depicts a first oligonucleotide primer comprising a 5′ end having a “tail” sequence or target noncomplementary region (“TNCR”) and a 3′ end in which is situated a sequence complementary to a template or target sequence, i.e., a target complementary region (“TCR”).

FIG. 2 depicts the extension of the first oligonucleotide primer using a polymerase, nucleotides, and so forth, and the target oligonucleotide to produce a first primer extension product (“first PEP”).

FIG. 3 depicts the extension of a second “reverse” primer using the first PEP as a template oligonucleotide to form a second PEP or the “amplification product.”

FIG. 4 depicts the method by which the amplification product is detected using a probe polynucleotide.

### DETAILED DESCRIPTION OF THE INVENTION

Gene variants are not only associated with adverse drug side effects. Variations in genes controlling patient drug response can also correlate with the inability of drugs to result in a successful therapeutic outcome. For example, Alzheimer's patients having the ApoE E4 subtype are less likely to benefit from the drug tacrine (Poirier et al. (1995) Proc. Natl Acad. Sci. USA 92:12260-4).

Inventions useful for assaying for particular polynucleotide sequences, whether based on SNPs, conserved sequences, or other features, have use in a wide variety of different applications. In addition to pharmacogenetic testing, such methods can be used in a forensic setting to identify the species or individual which was the source of a forensic specimen. Polynucleotide analysis methods can also be used in an anthropological setting. Paternity testing is another area in which the invention can be used, as is testing for compatibility between prospective tissue or blood donors and patients in need thereof, and in screening for hereditary disorders.

The invention taught herein can be used to study alterations of gene expression in response to a stimulus. Other applications include human population genetics, analyses of human evolutionary history, and characterization of human haplotype diversity.

The invention can also be used: to detect immunoglobulin class switching and hypervariable mutation of immunoglobulins; to detect polynucleotide sequences from contaminants or pathogens including bacteria, yeast and viruses; for HIV subtyping to determine the particular strains or relative amounts of particular strains infecting an individual; and can be done repeatedly to monitor changes in the individuals predominant HIV strains, such as the development of drug resistance or T cell tropism; and to detect single nucleotide polymorphisms, which may be associated with particular alleles or subsets of alleles. Over 1.4 million different single nucleotide polymorphisms (SNPs) in the human population have been identified (Nature 2001 409:928-933).

The invention can be used for mini-sequencing, and for detection of mutations. Any type of mutation can be detected, including without limitation SNPs, insertions, deletions, transitions, transversions, inversions, frame shifts, triplet repeat expansions, and chromosome rearrangements. The invention can be used to detect nucleotide sequences associated with increased risk of diseases or disorders, including cystic fibrosis, Tay-Sachs, sickle-cell anemia, etc.

The invention described herein is useful for any assay in which a sample can be interrogated regarding an amplification product from a target polynucleotide. Typical assays involve determining the presence of the amplification product in the sample or its relative amount, or the assays may be quantitative or semi-quantitative. Results from such assays can be used to determine the presence or amount of the target polynucleotide originally present in the sample.

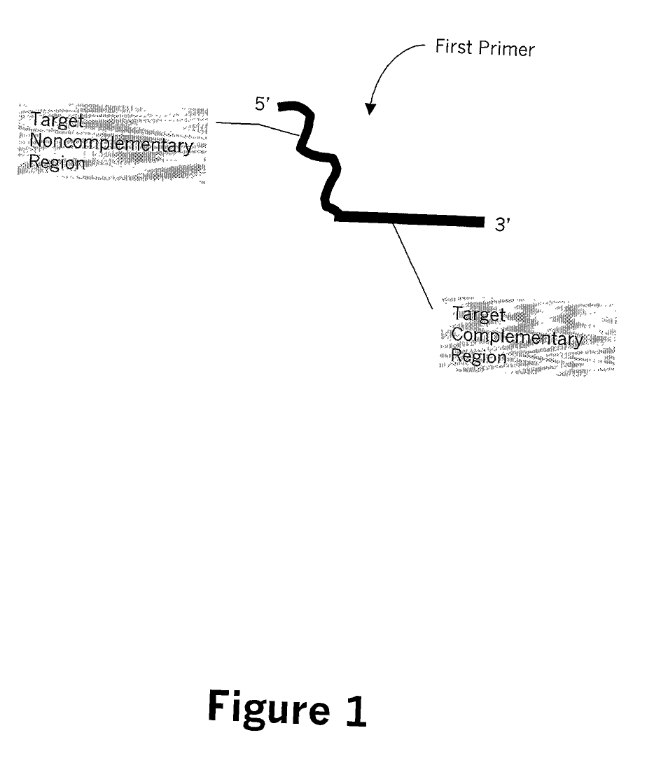

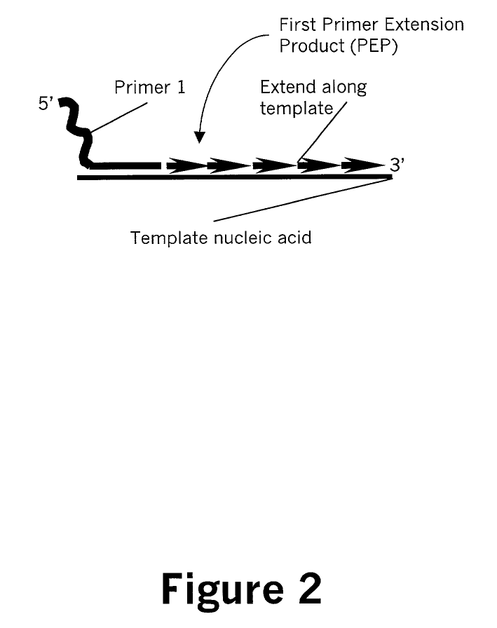

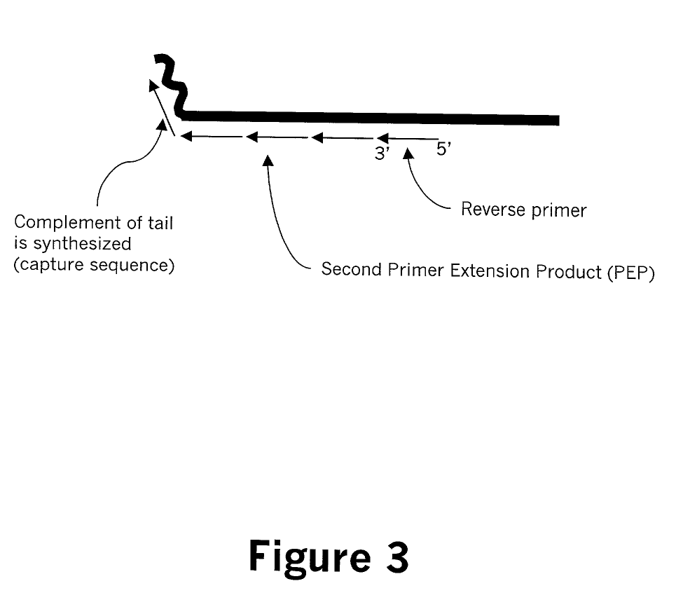

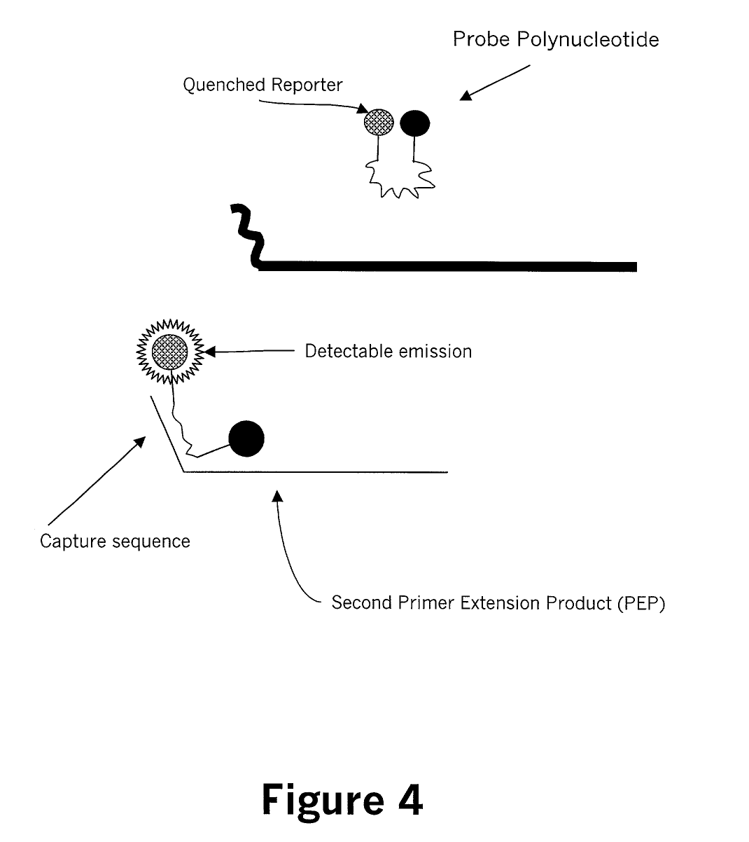

A sample suspected of containing the target polynucleotide is processed as described infra to prepare an amplification product. The amplification product-containing sample is then contacted with a first probe polynucleotide that comprises a molecular beacon. The probe polynucleotide binds to a “capture sequence” in the amplification product that does not naturally occur in the target or the amplification product. The capture sequence is introduced into the amplification product via primer-mediated extension from a template. Amplification reactions used to form the amplification product are also within the scope of the invention. A first primer is used to prime the synthesis of an oligonucleotide complementary to the target polynucleotide, forming a first primer extension product (“PEP”) (see FIG. 2). The first primer (see FIG. 1) comprises (a) a target complementary region (“TCR”) at its 3′ end that is at least in part complementary to at least a portion of the target polynucleotide and (b) a target noncomplementary region (“TNCR”) that does not normally occur adjacent to the TCR. A second primer, i.e., a “reverse” primer, is used to prime polynucleotide synthesis from the first PEP to form the second PEP (see FIG. 3). The second PEP is referred to herein as the “amplification product.” The second PEP comprises a sequence that is complementary to the TNCR in the first PEP; this TNCR-complementary sequence in the second PEP is referred to herein as the capture sequence. A label can be incorporated into the second PEP by any means, including by incorporation of a labeled nucleotide during extension from the second primer, but preferably the second primer itself comprises the label to increase specificity. The capture sequence in the amplification product binds specifically to a complementary sequence in the probe polynucleotide to form an amplification product detection complex (FIG. 4). In one embodiment, the probe polynucleotide is associated with a substrate and the amplification product detection product is localized to the substrate. Identification of the label in association with the substrate demonstrates that the amplification product was formed and the target polynucleotide was present in the sample.

The probe polynucleotide comprises a molecular beacon that, in turn, comprises an oligonucleotide sequence complementary to at least a portion of the capture sequence in the second PEP. The probe polynucleotide can be linked directly or indirectly to a substrate, and can be linked at any point in the polynucleotide that allows its effective use under assay conditions. The probe polynucleotide may also bind to an additional sequence in the second PEP distinct from the capture sequence, so long as it does not bind indiscriminately to any polynucleotide that lacks the capture sequence to a degree that significantly diminishes its effective use in the assay to be performed.

The molecular beacon comprises a quencher species and a fluorophore species, at least one of which is linked directly or indirectly at or in close proximity to a first end of the probe polynucleotide. The other of the quencher and fluorophore is linked directly or indirectly at or in close proximity to a second end of the probe polynucleotide or on a surface with which the probe polynucleotide is associated. The quencher and the fluorophore in this arrangement are of a type and are located such that the fluorescence emission from the fluorophore is quenched when the stem-loop structure is formed, and the fluorescence emission from the fluorophore is not quenched when the probe polynucleotide is hybridized to the capture sequence in the amplification product. In a variation, a self-quenching dye can be used that is both the fluorophore and quencher, and its location in the stem-loop structure can be varied as desired so that its fluorescence emission is either quenched or unquenched when the stem-loop structure is formed, with the converse occurring when the target polynucleotide is bound.

Upon hybridization of the capture sequence of the amplification product to the molecular beacon, at least a part of which is complementary to at least a part of the capture sequence in the amplification product, a change in the fluorescence characteristics of the molecular beacon occurs and can be detected.

The methods of the invention can all be performed in multiplex formats. A plurality of detectably distinct probe polynucleotides which specifically bind to corresponding distinct capture sequences in amplification products can be conjugated to the same substrate, or to a plurality of different distinguishable substrates. The separate binding of each distinct capture sequence to its corresponding probe polynucleotide can be detected by incorporating a detectably distinct label in each molecular beacon, by the location on a substrate at which each probe polynucleotide is located, by the conditions under which each capture sequence binds, or combinations of these methods. Multiplex methods are provided employing 2, 3, 4, 5, 10, 15, 20, 25, 50, 100, 200, 500, 1000, 2000, 5000, 10000, 25000, 50000, 100000 or more different probe polynucleotides which can be used simultaneously to assay for amplification products from corresponding distinct target polynucleotide.

Methods amenable to multiplexing, such as those taught herein, allow acquisition of greater amounts of information from smaller specimens or in shorter durations of time. The need for smaller specimens increases the ability to obtain samples from a larger number of individuals in a population to validate a new assay or simply to acquire data, as less invasive techniques are needed.

Where different substrates are included in a single multiplex assay, the different substrates can be encoded so that they can be distinguished. Any encoding scheme can be used; conveniently, the encoding scheme can employ one or more different fluorophores, which can be fluorescent semiconductor nanocrystals. High density spectral coding schemes can be used.

Before the present invention is described in detail, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a target polynucleotide” includes a plurality of target polynucleotides, reference to “a substrate” includes a plurality of such substrates, reference to “a probe polynucleotide” includes a plurality of probe polynucleotides, and the like.

Terms such as “associated,” “connected,” “attached,” “linked,” and “conjugated” are used interchangeably herein and encompass direct as well as indirect connection, attachment, linkage or conjugation unless the context clearly dictates otherwise.

When a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. When a value being discussed has inherent limits, for example if a component can be present at a concentration of from 0 to 100%, or if the pH of an aqueous solution can range from 1 to 14, those inherent limits are specifically disclosed. When a value is explicitly recited, it is to be understood that values, which are about the same quantity or amount as the recited value, are also within the scope of the invention. When a combination is disclosed, each subcombination of the elements of that combination is also specifically disclosed and is within the scope of the invention. Conversely, when different elements or groups of elements are disclosed, combinations thereof are also disclosed. When any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

Definitions

In describing the present invention, the following terms will be employed, and are intended to be defined as indicated below.

The term “nanoparticle” refers to a particle, generally a semiconductive or metallic particle, having a diameter in the range of about 1 nm to about 1000 nm, preferably in the range of about 2 nm to about 50 nm, more preferably in the range of about 2 nm to about 20 nm (for example about 2, 3, 4, 5, 6, 7, 8, 9, 10, 11, 12, 13, 14, 15, 16, 17, 18, 19, or 20 nm).

The terms “semiconductor nanoparticle” and “semiconductive nanoparticle” refer to a nanoparticle as defined above that is composed of an inorganic semiconductive material, an alloy or other mixture of inorganic semiconductive materials, an organic semiconductive material, or an inorganic or organic semiconductive core contained within one or more semiconductive overcoat layers.

The terms “semiconductor nanocrystal,” “quantum dot” and “Qdot™ nanocrystal” are used interchangeably herein to refer to semiconductor nanoparticles composed of an inorganic crystalline material that is luminescent (i.e., they are capable of emitting electromagnetic radiation upon excitation), and include an inner core of one or more first semiconductor materials that is optionally contained within an overcoating or “shell” of a second semiconductor material. A semiconductor nanocrystal core surrounded by a semiconductor shell is referred to as a “core/shell” semiconductor nanocrystal. The surrounding shell material will preferably have a bandgap energy that is larger than the bandgap energy of the core material and may be chosen to have an atomic spacing close to that of the core substrate. Suitable semiconductor materials for the core and/or shell include, but not limited to, the following: materials comprised of a first element selected from Groups 2 and 12 of the Periodic Table of the Elements and a second element selected from Group 16 (e.g., ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like); materials comprised of a first element selected from Group 13 of the Periodic Table of the Elements and a second element selected from Group 15 (GaN, GaP, GaAs, GaSb, InN, InP, InAs, InSb, and the like); materials comprised of a Group 14 element (Ge, Si, and the like); materials such as PbS, PbSe and the like; and alloys and mixtures thereof. As used herein, all reference to the Periodic Table of the Elements and groups thereof is to the new IUPAC system for numbering element groups, as set forth in the Handbook of Chemistry and Physics, 81^{st} Edition (CRC Press, 2000).

An SCNC is optionally surrounded by a “coat” of an organic capping agent. The organic capping agent may be any number of materials, but has an affinity for the SCNC surface. In general, the capping agent can be an isolated organic molecule, a polymer (or a monomer for a polymerization reaction), an inorganic complex, or an extended crystalline structure. The coat can be used to convey solubility, e.g., the ability to disperse a coated SCNC homogeneously into a chosen solvent, functionality, binding properties, or the like. In addition, the coat can be used to tailor the optical properties of the SCNC.

Thus, the terms “semiconductor nanocrystal,” “SCNC,” “quantum dot” and “Qdot™ nanocrystal” as used herein include a coated SCNC core, as well as a core/shell SCNC.

“Monodisperse particles” include a population of particles wherein at least about 60% of the particles in the population, more preferably about 75 to about 90, or any integer therebetween, percent of the particles in the population fall within a specified particle size range. A population of monodisperse particles deviates less than 10% rms (root-mean-square) in diameter, and preferably deviates less than 5% rms.

The phrase “one or more sizes of SCNCs” is used synonymously with the phrase “one or more particle size distributions of SCNCs.” One of ordinary skill in the art will realize that particular sizes of SCNCs are actually obtained as particle size distributions.

By “luminescence” is meant the process of emitting electromagnetic radiation (light) from an object. Luminescence results when a system undergoes a transition from an excited state to a lower energy state with a corresponding release of energy in the form of a photon. These energy states can be electronic, vibrational, rotational, or any combination thereof. The transition responsible for luminescence can be stimulated through the release of energy stored in the system chemically or added to the system from an external source. The external source of energy can be of a variety of types including chemical, thermal, electrical, magnetic, electromagnetic, and physical, or any other type of energy source capable of causing a system to be excited into a state higher in energy than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

The terms “polynucleotide,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, intemucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering finctional groups on the purine or pyrimidine base, which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide. Some or all of the residues in the polynucleotide can optionally be modified in one or more ways.

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′-H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine may be prepared by the method described by Switzer et al. (1993) Biochem. 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other nonnatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al. (1993), supra.

“Nucleic acid probe” and “probe” are used interchangeably and refer to a structure comprising a polynucleotide, as defined above, which contains a nucleic acid sequence that can bind to a corresponding target. The polynucleotide regions of probes may be composed of DNA, and/or RNA, and/or synthetic nucleotide analogs.

“Complementary” or “substantially complementary” refers to the ability to hybridize or base pair between nucleotides or nucleic acids, such as, for instance, between the two strands of a double stranded DNA molecule or between a polynucleotide primer and a primer binding site on a single stranded nucleic acid to be sequenced or amplified. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, Kanehisa (1984) Nucleic Acids Res. 12:203.

“Preferential binding” or “preferential hybridization” refers to the increased propensity of one polynucleotide to bind to a complementary polynucleotide in a sample as compared to noncomplementary polynucleotides in the sample or as compared to the propensity of the one polynucleotide to form an internal secondary structure such as a hairpin or stem-loop structure under at least one set of hybridization conditions.

Stringent hybridization conditions will typically include salt concentrations of less than about 1 M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art. Less stringent, and/or more physiological, hybridization conditions are used where a labeled polynucleotide amplification product cycles on and off a probe polynucleotide molecular beacon during a real-time assay which is monitored during PCR amplification such as a molecular beacon assay. Such less stringent hybridization conditions can also comprise solution conditions effective for other aspects of the method, for example, reverse transcription or PCR.

The terms “substrate” and “support” are used interchangeably and refer to a material having a rigid or semi-rigid surface.

A “homogeneous assay” is one that is performed without transfer, separation or washing steps. Thus, for example, a homogeneous single nucleotide polymorphism (“SNP”) assay involves the initial addition of reagents to a vessel, e.g., a test tube or sample well, followed by the detection of the results from that vessel. A homogeneous SNP assay can be performed anywhere in the vessel, for example in the solution, on the surface of the vessel or on beads or surfaces placed in the vessel. The detection system typically used is a fluorescence, chemiluminescence, or scintillation detection system.

“Multiplexing” herein refers to an assay or other analytical method in which multiple probe polynucleotides can be assayed simultaneously by using more than one SCNC, each of which has at least one different fluorescence characteristic (for example excitation wavelength, emission wavelength, emission intensity, FWHM (full width at half maximum peak height), or fluorescence lifetime). Multiplexing also includes assays or methods in which the combination of more than one SCNC having distinct emission spectra can be used to detect a single probe polynucleotide.

For example, two different preparations of SCNCs may have the same composition but different particle sizes, and thus differ in excitation and/or emission wavelength. Or, two different preparations may have the same particle size or particle size distribution but different composition, and thus also differ in excitation and/or emission wavelength. Different preparations having different compositions of SCNCs can have different fluorescent lifetimes, and thus their emission spectra can be distinguished even when they have the same emission wavelength and intensity, for example by sampling the emission from the encoded substance at different times after excitation. Differences in FWHM can be achieved for example by using SCNCs of different composition, or of the same composition but which are synthesized differently, or by mixing different SCNC “preparations” having overlapping emission peaks together to form a new preparation.

An SCNC having a known emission wavelength and/or intensity may be included with the SCNCs conjugated to the polynucleotide defined herein to provide an internal standard for calibrating the wavelength and/or intensity of the other SCNC(s) used in the conjugate.

“Optional” or “optionally” means that the subsequently described event or circumstance may or may not occur, and that the description includes instances where the event or circumstance occurs and instances in which it does not. For example, the phrase “optionally surrounded by a ‘coat’ of an organic capping agent” with reference to an SCNC includes SCNCs having such a coat, and SCNCs lacking such a coat.

The Sample

The portion of the sample comprising or suspected of comprising the target polynucleotide can be any source of biological material which comprises polynucleotides that can be obtained from a living organism directly or indirectly, including cells, tissue or fluid, and the deposits left by that organism, including viruses, mycoplasma, and fossils. The sample can also comprise a target polynucleotide prepared through synthetic means, in whole or in part. Typically, the sample is obtained as or dispersed in a predominantly aqueous medium. Nonlimiting examples of the sample include blood, urine, semen, milk, sputum, mucus, a buccal swab, a vaginal swab, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components), and a recombinant source, e.g. a library, comprising polynucleotide sequences.

The sample can be a positive control sample that is known to contain the target polynucleotide or a surrogate therefor. A negative control sample can also be used which, although not expected to contain the target polynucleotide, is suspected of containing it, and is tested in order to confirm the lack of contamination by the target polynucleotide of the reagents used in a given assay, as well as to determine whether a given set of assay conditions produces false positives (a positive signal even in the absence of target polynucleotide in the sample). The sample can be diluted, dissolved, suspended, extracted or otherwise treated to solubilize and/or purify any target polynucleotide present or to render it accessible to reagents which are used in an amplification scheme or to detection reagents. Where the sample contains cells, the cells can be lysed or permeabilized to release the polynucleotides within the cells. One step permeabilization buffers can be used to lyse cells which allow further steps to be performed directly after lysis, for example, a polymerase chain reaction.

The Target Polynucleotide and Amplification Products Produced Therefrom

The target polynucleotide can be single-stranded, double-stranded, or higher order, and can be linear or circular. Exemplary single-stranded target polynucleotides include mRNA, rRNA, tRNA, hnRNA, ssRNA or ssDNA viral genomes, although these single-stranded polynucleotides may contain internally complementary sequences and significant secondary structure. Exemplary double-stranded target polynucleotides include genomic DNA, mitochondrial DNA, chloroplast DNA, dsRNA or dsDNA viral genomes, plasmids, phage, and viroids. The target polynucleotide can be prepared synthetically or purified from a biological source. The target polynucleotide may be purified to remove or diminish one or more undesired components of the sample or to concentrate the target polynucleotide prior to amplification. Conversely, where the target polynucleotide is too concentrated for a particular assay, the target polynucleotide may first be diluted.

Following sample collection and optional nucleic acid extraction and purification, the nucleic acid portion of the sample comprising the target polynucleotide can be subjected to one or more preparative reactions. These preparative reactions can include in vitro transcription (IVT), labeling, fragmentation, amplification and other reactions. mRNA can first be treated with reverse transcriptase and a primer, which can be the first primer comprising the target noncomplementary region, to create cDNA prior to detection and/or further amplification; this can be done in vitro with purified mRNA or in situ, e.g., in cells or tissues affixed to a slide. Nucleic acid amplification increases the copy number of sequences of interest and can be used to incorporate a label into an amplification product produced from the target polynucleotide using a labeled primer or labeled nucleotide. A variety of amplification methods are suitable for use, including the polymerase chain reaction method (PCR), the ligase chain reaction (LCR), self sustained sequence replication (3SR), nucleic acid sequence-based amplification (NASBA), the use of Q Beta replicase, reverse transcription, nick translation, and the like, particularly where a labeled amplification product can be produced and utilized in the methods taught herein.

Where the target polynucleotide is single-stranded, the first cycle of amplification forms a primer extension product (PEP) complementary to the target polynucleotide. If the target polynucleotide is single-stranded RNA, a polymerase with reverse transcriptase is used in the first amplification to reverse transcribe the RNA to DNA, and additional amplification cycles can be performed to copy the PEPs. The primers for a PCR must, of course, be designed to hybridize to regions in their corresponding template that will produce an amplifiable segment; thus, each primer must hybridize so that its 3′ nucleotide is paired to a nucleotide in its complementary template strand that is located 3′ from the 3′ nucleotide of the primer used to prime the synthesis of the complementary template strand.

The target polynucleotide is typically amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, and enzymes having more than one type of polymerase activity. The polymerase can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfx, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H⁻ MMLV (SuperScript®), SuperScript® II, ThermoScript®, HIV- 1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pft, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions, optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different regions of a particular polynucleotide within the sample.

Where the amplification reaction comprises multiple cycles of amplification with a polymerase, as in PCR, it is desirable to dissociate the PEP(s) formed in a given cycle from their template(s). The reaction conditions are therefore altered between cycles to favor such dissociation; typically this is done by elevating the temperature of the reaction mixture, but other reaction conditions can be altered to favor dissociation, for example lowering the salt concentration and/or raising the pH of the solution in which the double-stranded polynucleotide is dissolved. Although it is preferable to perform the dissociation in the amplification reaction mixture, the polynucleotides may be first isolated using any effective technique and transferred to a different solution for dissociation, then reintroduced into an amplification reaction mixture for additional amplification cycles.

In one aspect of the invention, a first primer is used to amplify a specific target polynucleotide or region of a target polynucleotide. The first primer comprises at its 3′ end a target complementary region (“TCR”) that is complementary to a predetermined target polynucleotide, and a target noncomplementary region (“TNCR”), typically at its 5′ end. The TNCR is preferably introduced into the first primer during synthesis thereof, but could also be introduced via ligation. The TNCR preferably has a unique sequence that is not expected to occur in the sample, but at a minimum has a sequence that is not complementary to the sequence in the target polynucleotide that is 3′ of the sequence to which the TCR binds, and also does not otherwise occur in the amplification product produced by the amplification scheme employed. Hybridization of the first primer to the target polynucleotide and extension results in formation of a first PEP.

A second, or “reverse,” primer is provided that is complementary to a sequence in the first PEP which is 3′ from the TNCR. This second primer anneals to the first PEP and is capable of being extended in the opposite direction to form the second PEP. This second PEP comprises a capture sequence that is complementary to the TNCR in the first PEP. In one preferred embodiment, a label is incorporated into the second PEP to allow detection of a hybridization of the second PEP to the probe polynucleotide. Preferably this is done by incorporation of a label into the second primer. In this preferred method, a detectable label would be prepared if both first and second primers were present. In an alternative embodiment, label can be incorporated into the second PEP by incorporating a labeled nucleotide during the second extension reaction. It is possible in this alternative embodiment that spurious priming by the first PEP alone in two directions could lead to the preparation of a labeled second PEP and, possibly, a false positive result. Multiple cycles of such an amplification scheme can be performed. First PEPs produced in subsequent cycles from the first primer hybridizing to the second PEP and being extended may be shorter than first PEPs produced from the target polynucleotide where only a portion of the target polynucleotide is amplified.

It should be noted that “first primer” and “second primer” are relative terms as used herein. It is possible in some embodiments for the second primer to bind to a complement of the desired target polynucleotide, e.g., wherein the target polynucleotide is one strand of dsDNA. However, the only productive initiation of polynucleotide amplification occurs wherein the first primer binds and is extended to form the first PEP; a second PEP comprising the capture sequence is only formed by priming from that first PEP comprising the target noncomplementary region, i.e., from the second primer annealing to the first PEP at a position which is 3′ to the TNCR.

One allele or a subset of alleles of closely related sequences can be selectively assayed if at least one of the first and second primers is selective for that allele or subset of alleles. This is typically done by having the 3′ end of one of the primers overlap, typically by at least one to five nucleotides, a sequence variation specific to that allele or subset of alleles. This overlap allows the selective primer to bind preferentially and/or be extended from its exact match, with little or no extension from other alleles which may be present in the sample. In situation wherein the sequence variation being assayed is minimal, such as a single nucleotide polymorphism (“SNP”), the selective primer may hybridize to an incorrect allele; in a multiplex setting designed to detect both alleles of a SNP, such hybridization to incorrect alleles may adversely affect production of amplification product from the correct allele, an effect which may increase during amplification as the correct primer is diminished, leaving relatively higher concentrations of the mismatched primer. This effect can be minimized in various ways, including, for example, by incorporating a flanking primer located upstream, or 5′, from the selective primer in the target polynucleotide to increase the copy number of the template used for forming the first PEP. This flanking primer can have a lower melting temperature for binding to the target polynucleotide than the selective primer, so that the annealing temperature can be raised at some point during the amplification scheme to favor hybridization of the selective primer over the flanking primer.

This assay can be multiplexed, i.e., multiple distinct assays can be run simultaneously, by using different pairs of primers directed at different target polynucleotides. The different target polynucleotides can be, for example, unrelated targets, different alleles or subgroups of alleles from, or chromosomal rearrangements at, the same locus. This allows the quantitation of the presence of multiple target polynucleotides in a sample (e.g., specific genes in a cDNA library). Multiplex assays conducted in accordance with the invention disclosed herein require an ability to identify uniquely the distinct second PEP by using, e.g., a unique capture sequence in each first primer or a unique label in each second primer or incorporated into each second PEP.

Amplified target polynucleotides may be subjected to post-amplification treatments. For example, in some cases, it may be desirable to fragment the amplification products prior to hybridization with a polynucleotide array to provide segments that are more readily accessible and which avoid looping and/or hybridization to multiple probe polynucleotides. Fragmentation of the nucleic acids can be carried out by any method producing fragments of a size useful in the assay being performed; suitable physical, chemical and enzymatic methods are known in the art.

The amplification reaction can also be subjected to post-amplification treatments that remove undesirable components of the reaction mixture prior to detection. For example, unincorporated primers may be removed which might inhibit binding of the amplification product to the probe polynucleotide (unincorporated first primers, which contain the TNCR, can hybridize to the capture sequence and compete for binding of the capture sequence in the amplification product). Unincorporated primers can be removed, for example, by adding a single-stranded DNA nuclease (or other nuclease having a specificity for one or more components of the first primer) which can digest the unincorporated primers at a point in the amplification scheme at which the first and second PEPs should be hybridized in double-stranded form if they are present. The nuclease is preferably thermolabile so that the mixture can be heated to inactivate the nuclease so that the first and second PEPs can be dissociated without their digestion before detection. Alternatively, the first primer can be prepared with alternative bases which allow the first primer to be selectively destroyed; for example, the first primer may incorporate uracils which can be removed by digestion with uracil DNA glycosylase.

An amplification reaction can be performed under conditions that allow the probe polynucleotide to hybridize to the amplification product during at least part of an amplification cycle. When the assay is performed in this manner, real-time detection of this hybridization event can take place by monitoring for a change in fluorescence properties of the substrate that occurs upon such hybridization during the amplification. Alternatively, the amplification reaction may occur under conditions that do not allow such binding during cycling, for example, elevated temperature or in the absence of the probe polynucleotide, and the condition of the sample must be altered to allow detection to take place, for example, by lowering the temperature or by contacting the sample with the first or probe polynucleotide. The molecular beacon described below can be designed with the amplification reaction conditions in mind to either hybridize or not during an amplification cycle.

The Probe Polynucleotide

A probe polynucleotide is provided that comprises a molecular beacon that is a polynucleotide comprising a stem-loop, or hairpin, structure and a quenchable fluorophore.

Tyagi et al. (1996) Nature Biotech. 303-308 describes a molecular beacon oligonucleotide probe that is optically silent in solution but that fluoresces upon hybridization with a complementary target. Molecular beacons (MBs) are hairpin shaped where the loop portion is typically 15-30 nucleotides in length, but can be longer, and the stem portion is typically 4-7 nucleotides. A fluorophore can be attached to one end of the MB and a quencher can be attached to the other end. In the absence of a complement to the loop structure, the stem keeps the two moieties in close proximity to each other, causing the fluorescence to be quenched by energy transfer. When a complement to the loop is present, the loop sequence will form a hybrid with the target nucleic acid that is longer and more stable than the stem. The MB is thus linearized, causing the fluorophore and the quencher to move away from each other, leading to the restoration of fluorescence.

As disclosed herein, the probe polynucleotide can form a stem-loop structure wherein first and second complementary sequences thereof are capable of hybridizing to one another to form a stem and a third sequence located between the first and second sequences forms a loop in the absence of its complement. The third sequence is designed so that at least part of it is complementary to at least a part of the capture sequence, such that upon hybridization of the probe polynucleotide to the capture sequence, the stem-loop structure unfolds. The part of the probe polynucleotide that is complementary to the capture sequence can additionally comprise part of either or both the first and second complementary sequences, or can be located entirely within the loop sequence, and can be the entire loop sequence or only a portion therof. The part of the probe polynucleotide that is complementary to the capture sequence can be complementary to all or part of the capture sequence. The only requirement for the probe polynucleotide is that the stem-loop structure is formed under at least one set of hybridization conditions, and that the probe polynucleotide hybridizes preferentially to the capture sequence rather than forms the stem-loop structure under at least one set of hybridization conditions which occurs at some point during the assay being performed. Factors that can be modified in constructing such a probe polynucleotide include stem length and GC content, probe loop length and GC content, probe loop oligonucleotide spacer length, target concentration, target amplicon length, assay temperature, assay salt concentration, and assay incubation time.

The length of the probe polynucleotide is not critical, but typically is from 5-100 nucleotides in length and is chosen to provide suitably selective binding to the intended second primer extension product. The sequence of the loop region can be identical to the target noncomplementary region on a first PCR primer used in a PCR amplification reaction to produce the amplification product.

In one variation, the molecular beacon comprises a quencher and a fluorophore, at least one of which is linked at or nearer a first terminal end of the probe polynucleotide, directly or indirectly. The other of the quencher and fluorophore is directly or indirectly linked at or nearer a second terminal end of the probe polynucleotide. In an alternative variation, the other of the quencher or fluorphore is directly or indirectly linked to a surface, to which surface the probe polynucleotide is directly or indirectly linked. The quencher and the fluorophore in this arrangement are of a type and are located such that the fluorescence emission from the fluorophore is quenched when the stem-loop structure is formed, and the fluorescence emission from the fluorophore is not quenched when the probe polynucleotide is hybridized to the capture sequence. In a variation, a self-quenching dye can be used that is both the fluorophore and quencher, and its location in the stem-loop structure can be varied as desired so that its fluorescence emission is either quenched or unquenched when the stem-loop structure is formed, and unquenched or quenched, respectively, when the target polynucleotide is bound to the probe polynucleotide.

In yet another alternative embodiment, a self-quenchable fluorophore can be used that eliminates the need for a separate quencher in a molecular beacon (PCT Publ. No. WO 99/11813, published Mar. 11, 1999). The self-quenchable fluorophore can be quenched when the nucleotide to which it is attached is part of a duplex (e.g., BODIPY). Depending on whether the self-quenchable fluorophore is located in the stem or the loop region of the stem-loop structure, it may be quenched or unquenched either when bound to its corresponding capture sequence or when unbound and forming the stem-loop structure.

The probe polynucleotide can optionally be conjugated to a substrate. A substrate-bound probe polynucleotide can be synthesized directly on the substrate, or a probe polynucleotide can be synthesized separately from the substrate and then coupled to the substrate. Direct synthesis on the substrate may be accomplished by incorporating a monomer that is coupled to a subunit of the probe polynucleotide into a polymer that makes up or is deposited on or coupled to the substrate, and then synthesizing the remainder of the probe polynucleotide to incorporate that subunit. Alternatively, the substrate or its coating may include or be derivatized to include a functional group that can be coupled to a subunit of the probe polynucleotide for synthesis, or may be coupled directly to the complete probe polynucleotide. Suitable coupling techniques are well known in the art.

Hybridization of the second PEP to a probe polynucleotide conjugated to a substrate forms an amplification product assay complex. An amplification product assay array comprising a plurality of different probe polynucleotides conjugated to a substrate having different sequences hybridized to corresponding different capture sequences can also be prepared.

The Fluorophore

The fluorophore can be any substance that absorbs light of one wavelength and emits light of a different wavelength. Typical fluorophores include fluorescent dyes, semiconductor nanocrystals, lanthanide chelates, and a green fluorescent protein.

Exemplary semiconductor nanocrystals include those SCNCs described herein. Exemplary fluorescent dyes include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY FL, BODIPY FL-Br₂, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Exemplary lanthanide chelates include europium chelates, terbium chelates and samarium chelates.

The term “green fluorescent protein” refers to both native Aequorea green fluorescent protein and mutated versions that have been identified as exhibiting altered fluorescence characteristics, including altered excitation and emission maxima, as well as excitation and emission spectra of different shapes (Delagrave et al. (1995) Bio/Technology 13:151-154; Heim et al. (1994) Proc. Natl. Acad. Sci. USA 91:12501-12504; Heim et al. (1995) Nature 373:663-664).

The Quencher

The quencher can be any material that can quench at least one fluorescence emission from an excited fluorophore being used in the assay; that is, any material that can cause the excited fluorophore to emit detectably less energy under the assay conditions. A number of suitable quenchers are known in the art and are commercially available. Typical quenchers include DABCYL, BHQ-1, BHQ-2, BHQ-3, QSY 7, a metal nanoparticle, a semiconductor nanocrystal having a broad absorbance spectra and an emission wavelength outside the range being detected in the current assay, and a semiconductor nanocrystal having an absorbance spectra but no detectable emission.

### THE SUBSTRATE

The substrate can comprise a wide range of material, e.g., biological material, nonbiological material, organic material, inorganic material, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonate, or combinations thereof.

Substrates can be planar crystalline substrates such as silica based substrates (e.g., glass, quartz, or the like), or crystalline substrates used in, e.g., the semiconductor and microprocessor industries, such as silicon, gallium arsenide and the like.

Silica aerogels can also be used as substrates, and can be prepared by methods known in the art. Aerogel substrates may be used as free-standing substrates or as a surface coating for another substrate material.

The substrate can take any form and typically is a plate, slide, bead, pellet, disk, particle, strand, precipitate, membrane, optionally porous gel, sheets, tube, sphere, container, capillary, pad, slice, film, chip, multiwell plate or dish, optical fiber, or the like. Although typically the substrate takes an inanimate form, for some applications the substrate can be any form that is rigid or semi-rigid. The substrate may contain raised or depressed regions on which a probe polynucleotide is located. The surface of the substrate can be etched using well known techniques to provide for desired surface features, for example, trenches, v-grooves, mesa structures, or the like.

Surfaces on the substrate can be composed of the same material as the substrate or can be made from a different material, and can be coupled to the substrate by chemical or physical means. Such coupled surfaces may be composed of any of a wide variety of materials, for example, polymers, plastics, resins, polysaccharides, silica or silica-based materials, carbon, metals, inorganic glasses, membranes, or any of the above-listed substrate materials. In one embodiment, the surface will be optically transparent and will have surface Si—OH flnctionalities, such as those found on silica surfaces.

The substrate and/or its optional surface are chosen to provide appropriate optical characteristics for the synthetic and/or detection methods used. The substrate and/or surface can be transparent to allow the exposure of the substrate by light applied from multiple directions. The substrate and/or surface may be provided with reflective “mirror” structures to increase the recovery of light emitted by the semiconductor nanocrystal or other label. The substrate and/or its surface may also be coated to decrease the amount of spurious incident light.

The substrate and/or its surface is generally resistant to, or is treated to resist, the conditions to which it is to be exposed in use, and can be optionally treated to remove any resistant material after exposure to such conditions.

Oligonucleotides can be fabricated on or attached to the substrate by any suitable method, for example, the methods described in U.S. Pat. No. 5,143,854, PCT Publ. No. WO 92/10092, U.S. patent application Ser. No. 07/624,120, filed Dec. 6, 1990 (now abandoned), Fodor et al. (1991) Science 251:767-777 (1991), and PCT Publ. No. WO 90/15070). Techniques for the synthesis of these arrays using mechanical synthesis strategies are described in, e.g., PCT Publication No. WO 93/09668 and U.S. Pat. No. 5,384,261.

Still further techniques include bead-based techniques such as those described in PCT Appl. No. PCT/US93/04145 and pin-based methods such as those described in U.S. Pat. No. 5,288,514.

Additional flow channel or spotting methods applicable to attachment of oligonucleotides to a substrate are described in U.S. Pat. No. 5,384,261. Reagents are delivered to the substrate by either (1) flowing within a channel defined on predefined regions or (2) “spotting” on predefined regions. A protective coating such as a hydrophilic or hydrophobic coating (depending upon the nature of the solvent) can be used over portions of the substrate to be protected, sometimes in combination with materials that facilitate wetting by the reactant solution in other regions. In this manner, the flowing solutions are further prevented from passing outside of their designated flow paths.

Typical dispensers include a micropipette optionally robotically controlled, an ink-jet printer, a series of tubes, a manifold, an array of pipettes, or the like so that various reagents can be delivered to the reaction regions sequentially or simultaneously.

A MICROSPHERE SUBSTRATE. The substrate can be in the form of a microsphere. Polymeric microspheres or beads can be prepared from a variety of different polymers, including but not limited to polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers and epoxies. The materials have a variety of different properties with regard to swelling and porosity, which are well understood in the art. Preferably, the beads are in the size range of approximately 10 nm to 1 mm, and can be manipulated using normal solution techniques when suspended in a solution. The terms “bead,” “sphere,” “microbead” and “microsphere” are used interchangeably herein.

A plurality of such beads or mixtures of different bead populations can be immobilized on a planar surface such that they are regularly spaced in a chosen geometry using any suitable method. For example, beads can be immobilized by micromachining wells in which beads can be entrapped into the surface, or by patterned activation of polymers on the surface using light activation to cross-link single beads at particular locations. Suitable wells can be created by ablating circles in a layer of parylene deposited on a glass surface using a focused laser. The well dimensions are chosen such that a single bead can be captured per well and remain trapped when a lateral flow of fluid passes across the surface. For example, 7-micron wells can be readily used for analysis of beads about 4 microns to about 6 microns in diameter. This can be performed on the end of an optical fiber.

SPECTRALLYENCODED MICROSPHERES. Microspheres for use in the invention disclosed herein can be spectrally encoded through incorporation of SCNCs. See, e.g., U.S. Pat. No. 6,207,392 to Weiss et al., issued Mar. 27, 2001, International Pat. Publ. No. WO 00/17103 (inventors Bawendi et al.), published Mar. 30, 2000, and Han et al. (2001) Nature Biotech. 19:632-635. Conjugation of a probe polynucleotide to such an encoded microsphere produces an encoded bead conjugate. The desired fluorescence characteristics of the microspheres may be obtained by mixing SCNCs of different sizes and/or compositions in a fixed amount and ratio to obtain the desired spectrum, which can be determined prior to association with the microspheres. Subsequent treatment of the microspheres (through, for example, covalent attachment, co-polymerization, or passive absorption or adsorption) with the staining solution results in a material having the designed fluorescence characteristics.

A number of SCNC solutions can be prepared, each having a distinct distribution of sizes and compositions, to achieve the desired fluorescence characteristics. These solutions may be mixed in fixed proportions to arrive at a spectrum having the predetermined ratios and intensities of emission from the distinct SCNCs suspended in that solution. Upon exposure of this solution to a light source, the emission spectrum can be measured by techniques that are well established in the art. If the spectrum is not the desired spectrum, then more of the SCNC solution needed to achieve the desired spectrum can be added and the solution “titrated” to have the correct emission spectrum. These solutions may be colloidal solutions of SCNCs dispersed in a solvent, or they may be pre-polymeric colloidal solutions, which can be polymerized to form a matrix with SCNCs contained within.

The composition of the staining solution can be adjusted to have the desired fluorescence characteristics, preferably under the exact excitation source that will be used for the decoding. A multichannel auto-pipettor connected to a feedback circuit can be used to prepare an SCNC solution having the desired spectral characteristics, as described above. If the several channels of the titrator/pipettor are charged with several unique solutions of SCNCs, each having a unique excitation and emission spectrum, then these can be combined stepwise through addition of stock solutions.

Once the staining solution has been prepared, it can be used to incorporate a unique spectral code into a given bead population. If the method of incorporation of the SCNCs into the beads is absorption or adsorption, then the solvent that is used for the staining solution should be one that is suitable for swelling the microspheres, and can be selected based on the microsphere composition. Typical solvents for swelling microspheres include those in which the microsphere material is more soluble, for example dichloromethane, chloroform, dimethylformamide, tetrahydrofuran and the like. These can be mixed with a solvent in which the microsphere material is less soluble, for example methanol or ethanol, to control the degree and rate of incorporation of the staining solution into the material.

The microspheres swell when added to the staining solution and incorporate a plurality of SCNCs in the relative proportions that are present in the staining solution. After removal of the staining solution from the material, a nonswelling solvent is added, the material shrinks, or unswells, thereby trapping the SCNCs in the material. Alternatively, SCNCs can be trapped by evaporation of the swelling solvent from the material. After rinsing with a nonswelling solvent in which the SCNCs can be suspended, the SCNCs are trapped in the material, and can be retained by further use of a nonswelling solvent. Typical nonswelling solvents include hexane and toluene. The thus-encoded beads can be separated and exposed to a variety of solvents without a change in the emission spectrum under the light source. When the material used is a polymer bead, the material can be separated from the rinsing solvent by any suitable technique, for example, centrifugation, evaporation, fluidized bed drying, etc., or combined procedures, and can be redispersed into aqueous solvents and buffers through the use of detergents in the suspending buffer.

The staining procedure can also be carried out in sequential steps. A first staining solution can be used to stain the beads with one population of SCNCs. The beads can then be separated from the first staining solution and added to a second staining solution to stain the beads with a second population of SCNCs. These steps can be repeated until the desired spectral properties are obtained from the material when excited by a light source.

The SCNCs can be attached to the beads by covalent attachment as well as by entrapment in swelled beads, or can be coupled to one member of a binding pair the other member of which is attached to the beads. For instance, SCNCs are prepared by a number of techniques that result in reactive groups on the surface of the SCNC. See, e.g., Bruchez et al. (1998) Science 281:2013-2016, Chan et al. (1998) Science 281:2016-2018, Colvin et al. (1992) J. Am. Chem. Soc. 114:5221-5230, Katari et al. (1994) J. Phys. Chem. 98:4109-4117, Steigerwald et al. (1987) J. Am. Chem. Soc. 110:3046. The reactive groups present on the surface of the SCNCs can be coupled to reactive groups present on a surface of the material. For example, SCNCs which have carboxylate groups present on their surface can be coupled to beads with amine groups using a carbodiimide activation step.

Any cross-linking method that links a SCNC to a bead and does not adversely affect the properties of the SCNC or the bead can be used. In a cross-linking approach, the relative amounts of the different SCNCs can be used to control the relative intensities, while the absolute intensities can be controlled by adjusting the reaction time to control the number of reacted sites in total. After the beads are crosslinked to the SCNCs, the beads are optionally rinsed to wash away unreacted SCNCs.

A sufficient amount of fluorophore must be used to encode the beads so that the intensity of the emission from the fluorophores can be detected by the detection system used and the different intensity levels must be distinguishable, where intensity is used in the coding scheme but the fluorescence emission from the SCNCs or other fluorophores used to encode the beads must not be so intense to as to saturate the detector used in the decoding scheme.

The beads or other substrate to which one or more different known probe polynucleotides are conjugated can be encoded to allow rapid analysis of bead, and thus probe polynucleotide, identity, as well as allowing multiplexing. The coding scheme preferably employs one or more different SCNCs, although a variety of additional agents, including chromophores, fluorophores and dyes, and combinations thereof can be used alternatively or in combination with SCNCs. For organic dyes, different dyes that have distinguishable fluorescence characteristics can be used. Different SCNC populations having the same peak emission wavelength but different peak widths can be used to create different codes if sufficient spectral data can be gathered to allow the populations to be distinguished. Such different populations can also be mixed to create intermediate linewidths and hence more unique codes.

The number of SCNCs used to encode a single bead or substrate locale can be selected based on the particular application. Single SCNCs can be detected; however, a plurality of SCNCs from a given population is preferably incorporated in a single bead to provide a stronger, more continuous emission signal from each bead and thus allow shorter analysis time.

Different SCNC populations can be prepared with peak wavelengths separated by approximately 1 nm, and the peak wavelength of an individual SCNC can be readily determined with 1 nm accuracy. In the case of a single-peak spectral code, each wavelength is a different code. For example, CdSe SCNCs have a range of emission wavelengths of approximately 490-640 nm and thus can be used to generate about 150 single-peak codes at 1 nm resolution.

A spectral coding system that uses only highly separated spectral peaks having minimal spectral overlap and does not require stringent intensity regulation within the peaks allows for approximately 100,000 to 10,000,000 or more unique codes in different schemes.

A binary coding scheme combining a first SCNC population having an emission wavelength within a 490-565 nm channel and a second SCNC population within a 575-650 nm channel produces 3000 valid codes using 1-nm resolved SCNC populations if a minimum peak separation of 75 nm is used. The system can be expanded to include many peaks, the only requirement being that the minimum separation between peak wavelengths in valid codes is sufficient to allow their resolution by the detection methods used in that application.

A binary code using a spectral bandwidth of 300 nm, a coding-peak resolution, i.e., the minimum step size for a peak within a single channel, of 4 nm, a minimum interpeak spacing of 50 nm, and a maximum of 6 peaks in each code results in approximately 200,000 different codes. This assumes a purely binary code, in which the peak within each channel is either “on” or “off.” By adding a second “on” intensity, i.e., wherein intensity is 0, 1 or 2, the number of potential codes increases to approximately 5 million. If the coding-peak resolution is reduced to 1 nm, the number of codes increases to approximately 1×10¹⁰.

Valid codes within a given coding scheme can be identified using an algorithm. Potential codes are represented as a binary code, with the number of digits in the code corresponding to the total number of different SCNC populations having different peak wavelengths used for the coding scheme. For example, a 16-bit code could represent 16 different SCNC populations having peak emission wavelengths from 500 nm through 575 nm, at 5 nm spacing. A binary code 1000 0000 0000 0001 in this scheme represents the presence of the 500 nm and 575 nm peaks. Each of these 16-bit numbers can be evaluated for validity, depending on the spacing that is required between adjacent peaks; for example, 0010 0100 0000 0000 is a valid code if peaks spaced by 15 nm or greater can be resolved, but is not valid if the minimum spacing between adjacent peaks must be 20 nm. Using a 16-bit code with 500 to 575 nm range and 5 nm spacing between peaks, the different number of possible valid codes for different minimum spectral spacings between adjacent peaks is shown in Table 1.

TABLE 1
The number of unique codes with a binary 16-bit system.

| | 5 | 10 | 15 | 20 | 25 | 30 |
|---|---|---|---|---|---|---|
| Spectral Separation | nm | nm | nm | nm | nm | nm |
| Number of unique codes | 65535 | 2583 | 594 | 249 | 139 | 91 |

If different distinguishable intensities are used, then the number of valid codes dramatically increases. For example, using the 16-bit code above, with 15 nm minimum spacing between adjacent peaks in a code, 7,372 different valid codes are possible if two intensities, i.e., a ternary system, are used for each peak, and 38,154 different valid codes are possible for a quaternary system, i.e., wherein three “on” intensities can be distinguished.

Codes utilizing intensities require either precise matching of excitation sources or incorporation of an internal intensity standard into the beads due to the variation in extinction coefficient exhibited by individual SCNCs when excited by different wavelengths.

It is preferred that the light source used for the encoding procedure be as similar as possible (preferably of the same wavelength and intensity) to the light source that will be used for decoding. The light source may be related in a quantitative manner, so that the emission spectrum of the final material may be deduced from the spectrum of the staining solution.

Production of SCNCs

SCNCs can be made from any material and by any technique that produces SCNCs having emission characteristics useful in the methods, articles and compositions taught herein. The SCNCs have absorption and emission spectra that depend on their size, size distribution and composition. Suitable methods of production are disclosed in U.S. Pat. Nos. 6,048,616; 5,990,479; 5,690,807; 5,505,928; 5,262,357; 6,207,229; PCT Publication No. WO 99/26299 (published May 27, 1999; inventors Bawendi et al.); Murray et al. (1993) J. Am. Chem. Soc. 115:8706-8715; Guzelian et al. (1996) J. Phys. Chem. 100:7212-7219; Ridley et al. (1999) Science 286:746-749; Peng et al. (2001) 123:183-184; and Qu et al. (2001) Nano Lett. 1:333-337.

Examples of materials from which SCNCs can be formed include group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, Pb, Ge, Si, and other materials such as PbS, PbSe, and mixtures of two or more semiconducting materials, and alloys of any semiconducting material(s).

The composition, size and size distribution of the semiconductor nanocrystal affect its absorption and emission spectra. Exemplary SCNCs that emit energy in the visible range include CdS, CdSe, CdTe, ZnSe, ZnTe, GaP, and GaAs. Exemplary SCNCs that emit energy in the near IR range include InP, InAs, InSb, PbS, and PbSe. Exemplary SCNCs that emit energy in the blue to near-ultraviolet include ZnS and GaN. The size of SCNCs in a given population can be determined by the synthetic scheme used and/or through use of separation schemes, including for example size-selective precipitation and/or centrifugation. The separation schemes can be employed at an intermediate step in the synthetic scheme or after synthesis has been completed. For a given composition, larger SCNCs absorb and emit light at longer wavelengths than smaller SCNCs. SCNCs absorb strongly in the visible and UV and can be excited efficiently at wavelengths shorter than their emission peak. This characteristic allows the use in a mixed population of SCNCs of a single excitation source to excite all the SCNCs if the source has a shorter wavelength than the shortest SCNC emission wavelength within the mixture; it also confers the ability to selectively excite subpopulation(s) of SCNCs within the mixture by judicious choice of excitation wavelength.

The surface of the SCNC is preferably modified to enhance emission efficiency by adding an overcoating layer to form a “shell” around the “core” SCNC, because defects in the surface of the core SCNC can trap electrons or holes and degrade its electrical and optical properties. Addition of an insulating shell layer removes nonradiative relaxation pathways from the excited core, resulting in higher luminescence efficiency. Suitable materials for the shell include semiconductor materials having a higher bandgap energy than the core and preferably also having good conductance and valence band offset. Thus, the conductance band of the shell is desirably of a higher energy and the valence band is desirably of a lower energy than those of the core. For SCNC cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet may be used for the shell, for example ZnS, GaN, and magnesium chalcogenides, e.g., MgS, MgSe, and MgTe. For an SCNC core that emits in the near IR, materials having a bandgap energy in the visible, such as CdS or CdSe, or the ultraviolet may be used. Preparation of core-shell SCNCs is described in, e.g., Dabbousi et al. (1997) J. Phys. Chem. B 101:9463; Kuno et al. (1977) J. Phys. Chem. 106:9869; Hines et al. (1996) J. Phys. Chem. 100:468-471; PCT Publ. No. WO 99/26299; and U.S. Pat. No. 6,207,229 to Bawendi et al. issued Mar. 27, 2001. The SCNCs can be made further luminescent through overcoating procedures as described in Danek et al. (1996) Chem. Mat. 8(1):173-180, and Peng et al. (1997) J. Am. Chem. Soc. 119:7019-7029.

SCNCs are typically prepared in coordinating solvent, such as TOPO and trioctyl phosphine (TOP), resulting in the formation of a passivating organic layer on the surface of SCNCs with and without a shell. Such passivated SCNCs can be readily solubilized in organic solvents, for example toluene, chloroform and hexane. Molecules in the passivating layer can be displaced or modified to provide an outermost coating that adapts the SCNCs for use in other solvent systems, for example aqueous systems.

Alternatively, an outermost layer of an inorganic material such as silica can be added around the shell to improve the aqueous dispersibility of the SCNCs, and the surface of the silica can optionally be derivatized (Bruchez et al. (1998) Science 281:2013-2016 and U.S. Pat. No. 5,990,479 to Weiss et al.).

A displacement reaction may also be employed to modify the SCNC to improve the solubility in a particular organic solvent. For example, if it is desired to associate the SCNCs with a particular solvent or liquid, such as pyridine, the surface can be specifically modified with pyridine or pyridine-like moieties which are soluble or miscible with pyridine to ensure solvation. Water-dispersible SCNCs can be prepared as described in PCT Publ. No. WO 00/17655 (inventors Bawendi et al.), published Mar. 30, 2000 and U.S. Pat. No. 6,251,303 to Bawendi et al.

The surface layer of the SCNCs may be modified by displacement to render the SCNC reactive for a particular coupling reaction. For example, displacement of trioctylphosphine oxide (TOPO) moieties with a group containing a carboxylic acid moiety enables the reaction of the modified SCNCs with amine containing moieties to provide an amide linkage. For a detailed description of these linking reactions, see, e.g., U.S. Pat. No. 5,990,479 to Weiss et al.; Bruchez et al. (1998) Science 281:2013-2016, Chan et al. (1998) Science 281:2016-2018, Bruchez “Luminescent SCNCs: Intermittent Behavior and use as Fluorescent Biological Probes” (1998) Doctoral dissertation, University of California, Berkeley, and Mikulec “SCNC Colloids: Manganese Doped Cadmium Selenide, (Core)Shell Composites for Biological Labeling, and Highly Fluorescent Cadmium Telluride” (1999) Doctoral dissertation, Massachusetts Institute of Technology. The SCNC may be conjugated to other moieties directly or indirectly through a linker.

Examples of suitable spacers or linkers are polyethyleneglycols, dicarboxylic acids, polyamines and alkylenes. The spacers or linkers are optionally substituted with functional groups, for example hydrophilic groups such as amines, carboxylic acids and alcohols or lower alkoxy group such as methoxy and ethoxy groups. Additionally, the spacers will have an active site on or near a distal end. The active sites are optionally protected initially by protecting groups. Among a wide variety of protecting groups which are useful are FMOC, BOC, t-butyl esters, t-butyl ethers, and the like. Various exemplary protecting groups are described in, for example, Atherton et al., Solid Phase Peptide Synthesis, IRL Press (1989).

The Excitation Source

By exposing the encoded beads or other substrate prepared and described above to light of an excitation source, the SCNCs disposed in the material will be excited to emit light. This excitation source is of an energy capable of exciting at least one population of SCNCs used in the experiment to emit light and preferably chosen to be of higher energy than the shortest emission wavelength of the SCNCs used. Further, the excitation source can be chosen such that it excites a minimum number of SCNCs in the sample to produce detectable light. Preferably the excitation source will excite a sufficient number of different populations of SCNCs to allow unique identification of all the encoded materials used in the experiment. For example, using two different populations of beads having different ratios of red to blue SCNCs, it would not be sufficient to only excite the red emitting SCNCs, e.g., by using green or yellow light, of the sample in order to decode the beads. It would be necessary to use a light source comprising at least one wavelength that is capable of exciting the blue emitting and the red emitting SCNCs simultaneously, e.g., violet or ultraviolet. There may be one or more light sources used to excite the different populations of SCNCs simultaneously, or sequentially, but a given light source will only excite subpopulations of SCNCs that emit at lower energy than the light source, due to the absorbance spectra of the SCNCs.

In addition, if a lamp source is used, degradation of the lamp can result in changes in the excitation source, thereby compromising the codes.

Detection of SCNC Emission

An example of an imaging system for automated detection for use with the present methods comprises an excitation source, a monochromator (or any device capable of spectrally resolving the image, or a set of narrow band filters) and a detector array. The excitation source can comprise blue or UV wavelengths shorter than the emission wavelength(s) to be detected. This may be: a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm) or a HeCd laser; solid state diode lasers in the blue such as GaN and GaAs (doubled) based lasers or the doubled or tripled output of YAG or YLF based lasers; or any of the pulsed lasers with output in the blue.

The emitted light can be detected with a device that provides spectral information for the substrate, e.g., grating spectrometer, prism spectrometer, imaging spectrometer, or the like, or use of interference (bandpass) filters. Using a two-dimensional area imager such as a CCD camera, many objects may be imaged simultaneously. Spectral information can be generated by collecting more than one image via different bandpass, longpass, or shortpass filters (interference filters, or electronically tunable filters are appropriate). More than one imager may be used to gather data simultaneously through dedicated filters, or the filter may be changed in front of a single imager. Imaging based systems, like the Biometric Imaging system, scan a surface to find fluorescent signals.

A scanning system can be used in which the sample to be analyzed is scanned with respect to a microscope objective. The luminescence is put through a single monochromator or a grating or prism to spectrally resolve the colors. The detector is a diode array that then records the colors that are emitted at a particular spatial position. The software then recreates the scanned image.

Decoding Multiple Fluoroscence Emissions

When imaging samples labeled with multiple fluorophores, it is desirable to resolve spectrally the fluorescence from each discrete region within the sample. Such samples can arise, for example, from multiple types of SCNCs (and/or other fluorophores) being used to encode beads, from a single type of SCNC being used to encode beads but bound to a molecule labeled with a different fluorophore, or from multiple molecules labeled with different types of fluorophores bound at a single location. Decoding the spectral code of an encoded substrate can take place prior to, simultaneously with, or subsequent to determining whether a label from an amplification product is associated with the substrate.

Many techniques have been developed to solve this problem, including Fourier transform spectral imaging (Malik et al. (1996) J. Microsc. 182:133; Brenan et al. (1994) Appl. Opt. 33:7520) and Hadamard transform spectral imaging (Treado et al. (1989) Anal. Chem. 1:732A; Treado et al. (1990) Appl. Spectrosc. 44:1-4; Treado et al. (1990) Appl. Spectrosc. 44:1270; Hammaker et al. (1995) J. Mol. Struct. 348:135; Mei et al. (1996) J. Anal. Chem. 354:250; Flateley et al. (1993) Appl. Spectrosc. 47:1464), imaging through variable interference (Youvan (1994) Nature 369:79; Goldman et al. (1992) Biotechnol. 10:1557), acousto-optical (Mortensen et al. (1996) IEEE Trans. Inst. Meas. 45:394; Turner et al. (1996) Appl. Spectrosc. 50:277) or liquid crystal filters (Morris et al. (1994) Appl. Spectrose. 48:857) or simply scanning a slit or point across the sample surface (Colarusso et al. (1998) Appl. Spectrosc. 52:106A), all of which are capable of generating spectral and spatial information across a two-dimensional region of a sample.

One-dimensional spectral imaging can easily be achieved by projecting a fluorescent image onto the entrance slit of a linear spectrometer. In this configuration, spatial information is retained along the y-axis, while spectral information is dispersed along the x-axis (Empedocles et al. (1996) Phys. Rev. Lett. 77(18):3873). The entrance slit restricts the spatial position of the light entering the spectrometer, defining the calibration for each spectrum. The width of the entrance slit, in part, defines the spectral resolution of the system.

Two-dimensional images can be obtained by eliminating the entrance slit and allowing the discrete images from individual points to define the spatial position of the light entering the spectrometer. In this case, the spectral resolution of the system is defined, in part, by the size of the discrete images. Since the spatial position of the light from each point varies across the x-axis, however, the calibration for each spectrum will be different, resulting in an error in the absolute energy values. Splitting the original image and passing one half through a dispersive grating to create a separate image and spectra can eliminate this calibration error. With appropriate alignment, a correlation can be made between the spatial position and the absolute spectral energy.

To avoid ambiguity between images that fall along the same horizontal line, a second beam-splitter can be added, with a second dispersive element oriented at 90 degrees to the original. By dispersing the image along two orthogonal directions, it is possible to unambiguously distinguish the spectra from each discrete point within the image. The spectral dispersion can be performed using gratings, for example holographic transmission gratings or standard reflection gratings. For example, using holographic transmission gratings, the original image is split into 2 (or 3) images at ratios that provide more light to the spectrally dispersed images, which have several sources of light loss, than the direct image. This method can be used to spectrally image a sample containing discrete point signals, for example, in high throughput screening of discrete spectral images such as single molecules or ensembles of molecules immobilized on a substrate, and for highly parallel reading of spectrally encoded beads. The images are then projected onto a detector and the signals are recombined to produce an image that contains information about the amount of light within each band-pass.

Alternatively, techniques for calibrating point spectra within a two-dimensional image are unnecessary if an internal wavelength reference (the “reference channel”) is included within the spectrally encoded material. The reference channel is preferably either the longest or shortest wavelength emitting fluorophore in the code. The known emission wavelength of the reference channel allows determination of the emission wavelengths of the fluorophores in the dispersed spectral code image. In addition to wavelength calibration, the reference channel can serve as an intensity calibration where coding schemes with multiple intensities at single emission wavelengths are used. Additionally, a fixed intensity of the reference channel can also be used as an internal calibration standard for the quantity of label bound to the surface of each bead.

In one system for reading spectrally encoded beads or materials, a confocal excitation source is scanned across the surface of a sample. When the source passes over an encoded bead, the fluorescence spectrum is acquired. By raster-scanning the point-excitation source over the sample, all of the materials within a sample can be read sequentially.

SCNC codes can be detected using the microscope-based system for visualizing fluorescent images described above and in U.S. application Ser. No. 09/827,076, entitled “Two-dimensional Spectral Imaging System” by Empedocles et al, filed Apr. 5, 2001.

SNP Detection [Using Encoded Beads]

Allele-specific first primers are synthesized that each comprise at its 3′ end an allele-specific target complementary region. that is complementary to a predetermined target polynucleotide. Each allele-specific first primer also comprises a distinct target noncomplementary region, typically at its 5′end (see, FIG. 1) that is not complementary to any of the alleles to be detected. The same second, i.e., “reverse,” primer can be used for all alleles. Each allele-specific first primer hybridizes to the allele-specific target and is extended by a polymerase to form an allele-specific first primer extension product (PEP). Each allele-specific first PEP comprises the respective allele-specific target noncomplementary region.

Each allele-specific probe oligonucleotide (molecular beacon) comprises a loop region that includes an allele-specific capture sequence complementary oligonucleotide chain, and a reporter fluorophore that is spectrally distinct from other reporter fluorophores to be incorporated into the assay.

In one alternative embodiment, each allele-specific probe oligonucleotide is linked directly or indirectly through a linker to a spectrally encoded bead.

A batch of spectrally encoded beads with known fluorescence characteristics is created by imbedding semiconductor nanocrystals into microspheres as described above. The beads can be prepared having a functional chemical group (e.g., a carboxyl group) on the surface. Allele-specific probe polynucleotides are prepared, e.g., chemically synthesized, to have a predetermined nucleotide chain that can hybridize to the respective allele-specific capture sequence in the second PEP product and a functionalized linker (e.g., a 3′ or 5′ amino linker) that allows it to be attached covalently to the spectrally encoded beads, and then purified. The probe polynucleotide is chemically conjugated to the surface of the spectrally encoded beads to form an encoded bead conjugate using any suitable method (e.g., covalent attachment of an amino-labeled oligonucleotide onto carboxylated beads in the presence of EDC).

The encoded bead conjugates are then incubated with the amplification reaction mixture. If the target polynucleotide was present in the sample and the second PEP comprising the allele-specific capture sequence was produced during the amplification reaction, the second PEP will hybridize to the respective allele-specific probe polynucleotide on the surface of the encoded bead conjugate (or other substrate) during this incubation to form an amplification product detection complex. The fluorescence emission from the fluorophore in the molecular beacon can then be detected and optionally quantitated.

This assay can be multiplexed, i.e., multiple distinct assays can be run simultaneously, by using different pairs of allele-specific first primers to detect multiple single nucleotide polymorphisms (SNPs) within a sample. Allele-specific priming can be used to distinguish between alleles of individual SNPs in the same reaction. The allele-specific first primer comprises a 3′ end designed to overlap the location of the SNP by 1-5 nucleotides. If the allele-specific first primer is completely complementary to the target SNP sequence, it will anneal and extend. If the allele-specific first primer has a mismatch at the location of the SNP, it may partially anneal but cannot be extended, or is extended at a much lower efficiency.

The allele-specific first primer is selective, i.e., each allele-specific first primer comprises a unique target noncomplementary region. Batches of beads containing different spectral codes are separately conjugated to different allele-specific probe polynucleotides specific for corresponding different allele-specific capture sequences. When a first primer is extended, its unique target noncomplementary region becomes incorporated into that first PEP. The second primer can be 5′ end-labeled (e.g., with biotin or fluorescein) during synthesis, and can be used in conjunction with the selective first primer to perform an allele-specific PCR. A target polynucleotide containing the noncomplementary allele will not be significantly amplified, due to the mismatch under the primer sequence. By using a different unique target noncomplementary region in each allele-specific first primer, it can be ensured that a unique capture sequence will be incorporated into the second PEP.

Use of the Methods With Microarrays

Microarray slides attached to probe polynucleotides can be prepared as described at www.nhgri.nih.gov/DIR/Microarray/fabrication.html, also set forth in U.S. patent application Ser. No. 09/675,528 by Empedocles et al. entitled “Microarray Methods Utilizing Semiconductor Nanocrystals”, filed Sep. 29, 1900. Further guidance on fabrication, sample labeling and conditions for hybridization using microarrays is provided, for example, by Bittner et al. (2000) Nature 406:536-540; Khan et al. (1999) Electrophoresis 20:223-9; Duggan. (1999) Science 283:83-87; and DeRisi et al. (1996) Nature Genet. 14:457-60.

In a typical microarray experiment, the sample suspected of containing the target polynucleotide is treated to form the respective amplification product. The amplification products are optionally mixed with blockers, for example tRNA, Cot1 DNA, or purified repeat sequences such as LINE or Alu sequences, or mixtures thereof. Nonnucleotide blocking agents can also be used, including proteins, for example BSA, and detergents. This mixture is then incubated with the microarray slides comprising the probe oligonucleotide. The slides are then rinsed.

The microarray can then be scanned with a laser scanner having an excitation source and emission filters appropriate for the particular SCNC(s) or other fluorophore used, or the microarray can be scanned with a wide-field imaging scanner having appropriate excitation and emission filters.

Kits

Kits comprising reagents useful for performing the methods of the invention are also provided. In one embodiment, a kit comprises a probe polynucleotide and first and second primers for preparing a target-specific amplification product. The probe polynucleotide can be provided attached to a substrate, which can be an encoded bead comprising a first spectral code comprising a first semiconductor nanocrystal and first fluorescence characteristics. The probe polynucleotide can bind to the amplification product produced from the target polynucleotide, and a sample may be assayed for the presence of such a target polynucleotide or amplification product produced therefrom using the components of the kit. In an alternative embodiment, the probe polynucleotide can be provide attached to a planar substrate in the form of a microarray.

The components of the kit are retained by a housing. Instructions for using the kit to perform a method of the invention are provided with the housing, and may be located inside the housing or outside the housing, and may be printed on the interior or exterior of any surface forming the housing which renders the instructions legible. The kit may be in multiplex form, containing pluralities of one or more different probe polynucleotides, substrates and/or pairs of primers. The substrate may comprise a plurality of probe polynucleotides of different sequence for performing a plurality of individual assays thereon such as a microarray, or a plurality of different beads can be provided for a multiplexed assay wherein each of the different beads comprises a different probe polynucleotide for binding to a corresponding different amplification product.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Example 1

### Spectrally Encoding and Functionalizing Microspheres

A. The following experiment was performed to prepare encoded and finctionalized microspheres via a heat-swelling method and Dextran Biotin coating.

MATERIALS: 10 μm Bangs COOH functionalized beads 10% solid (Bangs Lab); 10 mM phosphate-buffered saline (PBS), pH 7.4 (Sigma); 10 mM PBS/1% bovine serum albumin (BSA); dihydrolipoic acid (DHLA)-derivatized SCNCs (U.S. Pat. No. 6,207,229 to Bawendi et al.); dextran biotin, 10 mg/mL (Sigma Cat#B5512, Lot#81H0080); and streptavidin, 10 mg/mL (Pierce Cat#21 125B, Lot#AH41661).

PROTOCOL: The beads were washed 3 times with PBS buffer and resuspended in PBS to make a 5% (w/v) bead solution. The bead solution was heated in a heat block up to about 60° C. with constant mixing. DHLA SCNCs were added (the amount added depended on the intended intensity for the particular beads); The mixture of beads and SCNC was incubated for 5-10 min. at 60° C. The encoded beads were washed 3 times with PBS and resuspended in PBS. The intensity and uniformity of encoded beads was checked using a FACScan and by microscopic inspection. Dextran Biotin (10 mg/mL) was added to the encoded beads solution and the resulting mixture was incubated at room temperature overnight with constant mixing. The biotin dextran-coated encoded beads were washed with PBS and resuspended in PBS/BSA. Streptavidin (final conc. of 5 mg/ml) was added to the dextran-biotin-coated encoded beads and incubate at room temperature for 3-4 hours. The streptavidin-conjugated encoded beads thus formed were washed with PBS and resuspended in PBS/BSA. The streptavidin-conjugated encoded beads are ready for use to attach a biotinylated probe polynucleotide of choice.

B. Encoding beads with DHLA SCNCs with BSA absorption and functionalizing beads with Streptavidin by Maleimide conjugation

MATERIALS: 10 μm Bangs COOH beads 10% solid (Bangs Lab); 10 mM phosphate buffered saline/1% bovine serum (BSA), pH 7.4; dihydrolipoic acid (DHLA) SCNCs of different emission colors (U.S. Pat. No. 6,251,300); Sulfo-SMCC (Cat#22322, Lot#AF40301) or Sulfo-SMPB (Pierce); conjugation buffer and elution buffer for NAP5 column comprising 0.1 M Sodium Phosphate, 0.15 M NaCl, 10 mM EDTA, pH 7.2; NAP5 column equilibration buffer: 10 mM Sodium Phosphate, pH 6.8; NAP5 (Sephadex G25 resin, Pharmacia (Cat#17-0853-02, Lot#278694)); Streptavidin (Pierce Cat# 21125B, Lot#AH41661); and 2-Iminothiolane (Sigma Cat#I-256, Lot#128H1085).

PROTOCOL: Preparation of SCNC-coded beads. The beads were washed 3 times with PBS. To the washed beads was added 1% BSA/PBS solution and the beads were incubated at room temperature overnight with constant mixing. The BSA-coated beads thus formed were washed 3 times with PBS. DHLA SCNCs were added and the resulting mixture was incubated for 15-30 min at room temperature with constant mixing. The excess DHLA SCNCs was washed off with PBS and the BSA-coated encoded beads were resuspended in conjugation buffer. BSA-coated encoded beads can be coated with another layer of BSA by incubating these beads with 1% BSA for several hours. 2-iminothiolane (20mg/mL) was added to the BSA-coated encoded beads mixture and the resulting reaction mixture was incubated at room temperature for 1-2 hr. with constant mixing. The beads thus formed were 3 times and resuspended in conjugation buffer.

Maleimide activation of Streptavidin. To 20 mg/mL solution of streptavidin (in conjugation buffer) was added Sulfo-SMCC or Sulfo-SMPB (6 mg/mL in water). The resultant mixture was incubated at room temperature for 30 min. The maleimide-activated streptavidin thus formed was purified using a NAP5 column with 10-15 mL equilibration buffer (pre-loading) and with 1-2 mL elution buffer (post-loading).

The SCNC-encoded beads and the maleimide-activated streptavidin were mixed and reacted at room temperature for 2-3 hrs. The streptavidin-conjugated encoded beads thus formed were washed with PBS and resuspended in PBS/BSA.

### Example 2

### Homogeneous Genotyping Assay

Assay Concept. An allele specific PCR is performed using two allele-specific first primers and a common second primer. The two molecular beacons specific for two different capture sequences are included in the reaction mixture during the PCR reaction. If the sample being amplified only contains allele A, only the allele A-specific first primer participants in the amplification reaction. Every cycle after the first cycle of the PCR, the second primer will anneal to the first PEP and be extended to from a second PEP comprising a capture sequence that is complementary to the sequence in the respective probe oligonucleotide. The complement of the target NCR is a newly created sequence in the solution. The presence of the transcribed tag sequence is mediated by the presence or absence of the specific SNP sequence for which the generic sequence-tagged primer was designed. Molecular beacons having detectably distinct flurophores are used. The length of the stem and loop in the molecular beacons do not have to be calibrated to opened only using a complementary sequence. Instead, the beacons are present during the PCR amplification reaction. Under the temperatures required for PCR amplification, the beacons will be maintained in an unfolded state. The heat energy at the >60C annealing temperature is sufficient to eliminate the secondary structure within the beacon. In alternative embodiments, the annealing temperature and/or the melting temperature of the MBs can be varied so that the MB can fold in the absence of its capture sequence during PCR. After the last cycle of PCR amplification, the amplification solution is cooled in the thermal cycler in a controlled fashion. If the sequence complementary to the beacon is present, the beacon will hybridize and thus will remain open and the fluorophore will be unquenched and, thus, detectable. If the complementary sequence is not present, then the decreased temperature within the solution will allow the beacon to refold, resulting in the quenching of the fluorophore. The reaction is homogeneous, i.e., all of the reactants are initially assembled into the solution, with no further addition of reagents needed. The PCR amplification is performed, and then the solution is scanned in, e.g., a fluorescence plate reader, with no further addition of reagents needed. The fluorescent signal intensity for each of the two fluorophores in the MBs specific for the two alleles is measured, and the relative signals quantitated.

Protocol. A SNP to be tested is identified. Preferably, a minimum of about 20 nucleotides on each side of the polymorphic site needs to be known for primer design; 40 nucleotides are more preferred. A sequence-specific first primer is designed for allele A of the SNP marker. This first primer is designed such that (1) it specifically primes the intended target sequence (allele A), (2) it does not significantly prime the target sequence for allele B, (3) it is approximately 13-17 nucleotides in length, depending on the GC content/Tₘ of the target (typically, it will be shorter than conventional PCR primers so that the specificity of priming is improved); (4) the 3′ end of the primer overlaps the polymorphic site anywhere from 1-4 nucleotides, depending on the specific sequence and an empirical determination of the requirements for specificity; (5) the 5′ end of the primer is synthesized to include a predetermined sequence that is not complementary to allele A and that is an allele-specific capture sequence of approximately 18-24 nucleotides long; and (6) the 3′ ends are as AT rich as possible.

The same steps are applied to create an allele-specific first primer for allele B, except the primer is complementary to allele B, and a distinct capture sequence is used. This capture sequence is designed to have little identity with the target NCR for allele A to avoid cross-reaction during the assay.

A common second primer is designed in a region of conserved sequence between the alleles. This primer is typically 17-24 nucleotides long, and is designed to give robust amplification, but have little chance for non-specific amplification with the allele-specific first primers.

A first allele-specific probe oligonucleotide sequence is designed with a loop sequence that can bind to the first allele-specific capture sequence like the target NCR in the allele A-specific first primer. However, while the sequence can be the same, it need not be exactly the same length. For instance, the target NCR sequence in the first primer could be a 24-mer, and the loop sequence in the probe oligonucleotide could be a 22-mer. The 5′ end of the probe oligonucleotide contains fluorescein (green) and the 3′ end contains a quencher for FITC.

A second allele-specific probe oligonucleotide sequence is designed similar to the first allele-specific probe oligonucleotide, but specific for allele B. The 5′ end of the probe oligonucleotide contains a red fluorescent dye and the 3′ end contains a quencher for this red dye.

These primers are placed into a PCR mixture, containing the following components:

TABLE 2
PCR Mixture Components

| Reagent | Concentration |
|---|---|
| Allele A-specific first primer | 0.1 μM |
| Allele B-specific first primer | 0.1 μM |
| Allele A-specific probe oligonucleotide | 0.4 μM |
| Allele B-specific probe oligonucleotide | 0.4 μM |
| Reverse Primer | 1.0 μM |
| AmpliTaq ®Stoffel fragment buffer | 1X |
| AmpliTaq ®Stoffel Enzyme | 0.05 Units/μL |
| DNTP | 0.2 mM each |
| MgCl₂ | 2.5 mM |
| DNA sample to be analyzed | 0.5-2 ng/amplification |
| Water | QS volume |

The PCR amplification is performed 10 μL/well in a 384 well plate.

TABLE 3
PCR protocol

(94 ° C., 30 sec), (62 ° C., 30 sec), (72 ° C., 30 sec) for 2 cycles
(94 ° C., 30 sec), (68 ° C., 30 sec), (72 ° C., 30 sec) for 38 cycles
(72 ° C., 7 minutes)
temp ramp 72 ° C. □ 35° C., 10 minutes
Total PCR amplification time less than about 2 hours

The plate is then read in a fluorescent plate reader, with two channels optimized for the two colors of fluorophores being used. Results for each well are reported as the signal intensity for the green channel and signal intensity for the red channel: green signal alone indicates AA genotype; red signal alone indicates BB genotype; and green and red signals of nearly equal proportion indicate an AB genotype.

The following controls can be included in this assay: (a) PCR contamination control, in which no genomic DNA sample is added; (b) a well containing a negative amplification control, in which a crucial component necessary for the PCR (e.g., the polymerase) is not added to test the stability of the dye-conjugated beacons, and test for the refolding of beacons after the cooling reaction (signal observed in this well can be subtracted as true background); (c) a well in which a chemical denaturant (e.g., weak NaOH) is added. If the condition is set up so that the beacon cannot fold, 100% of the possible signal in each well could be measured directly, since all of the molecules of fluor would be unquenched. This allows both absolute measurements of how much beacon is present, as well as a comparison between the signal intensities of the two colors for the two beacons. This can be used as a valuable QC assay for characterizing the reagents; and (d) positive controls comprising two oligonucleotides, which are complementary to the loops of the two probe oligonucleotides, can be used, singly or in pairs. The nature of the positive control can be varied at will: one or both oligo sequences, low oligo concentration, high oligo concentration, 1:1 mixture of the two sequences, varying ratios of the two oligos, etc.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

What is claimed is:

1. A method for assaying a sample for an amplification product from a target polynucleotide, comprising:
contacting the sample which is suspected of containing the amplification product with a probe polynucleotide;
wherein the amplification product is a polynucleotide and comprises a capture sequence whose complement is not present in the unamplified target polynucleotide at the same position;
wherein the probe polynucleotide comprises a molecular beacon comprising first and second complementary regions and a third region located between the first and second complementary regions;
wherein the molecular beacon can form a stem-loop structure in which the first and second complementary regions hybridize to each other to form a stem and the third region forms a loop;
wherein at least part of the third region is complementary to at least part of the capture sequence in the amplification product, such that the probe polynucleotide can preferentially hybridize to the amplification product and thereby disrupt formation of the stem-loop structure under at least one set of hybridization conditions;
wherein the probe polynucleotide is linked to a first quencher and to a first fluorophore, wherein the first quencher and first fluorophore are located such that the first quencher can quench a fluorescence emission from the first fluorophore either under a first hybridization state when the probe polynucleotide is not hybridized to the amplification product and the stem-loop structure is formed or under a second hybridization state when the probe polynucleotide is hybridized to the amplification product and the stem-loop structure is not formed, but not under both hybridization states;
wherein the contacting takes place under conditions in which the probe polynucleotide can hybridize to the amplification product, if present;
exciting the first fluorophore by applying a light source to the sample; and
determining the fluorescence emission from the first fluorophore.

2. The method of claim 1, wherein the first fluorophore is a semiconductor nanocrystal.

3. The method of claim 2, wherein the semiconductor nanocrystal comprises a core selected from the group consisting of ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, GaN, GaP, GaAs, GaSb, InN, InP, InAs, InSb, AlAs, AlP, AlSb, AlS, Ge, Si, Pb, PbS, PbSe, an alloy thereof, and a mixture thereof.

4. The method of claim 3, wherein the core is CdSe.

5. The method of claim 1, wherein the first fluorophore is a dye.

6. The method of claim 5, wherein the dye is also the first quencher.

7. The method of claim 1, wherein the first quencher quenches the fluorescence emission from the first fluorophore under the first hybridization state.

8. The method of claim 1, wherein the first quencher quenches the fluorescence emission from the first fluorophore under the second hybridization state.

9. The method of claim 1, wherein the first quencher is selected from DABCYL, BHQ-1, BHQ-2, BHQ-3, a metal nanoparticle, and a semiconductor nanocrystal.

10. The method of claim 1, wherein the sample is assayed for the presence of the amplification product.

11. The method of claim 10, wherein a result of the assay for the presence of the amplification product is used to determine the presence of the target polynucleotide in the sample prior to production of the amplification product.

12. The method of claim 1, wherein the sample is assayed to determine the amount of the amplification product.

13. The method of claim 12, wherein a result of the assay to determine the amount of the amplification product is used to determine the amount of the target polynucleotide in the sample prior to production of the amplification product.

14. The method of claim 1, wherein the amplification product is produced at a detectably higher level from a first allele of a locus having at least two alleles.

15. The method of claim 14, wherein the at least two alleles differ by a single nucleotide.

16. The method of claim 14, wherein the first allele is an allele associated with an increased risk of a disease or disorder.

17. The method of claim 14, wherein the first allele comprises an inactivating mutation of a tumor suppressor gene.

18. The method of claim 14, wherein the first allele comprises an activating mutation of a cellular oncogene.

19. The method of claim 1, wherein the probe polynucleotide is attached to a solid substrate.

20. The method of claim 19, wherein the solid substrate is a microsphere.

21. The method of claim 20, wherein the microsphere comprises a spectral code comprising a first semiconductor nanocrystal having first fluorescence characteristics.

22. The method of claim 19, wherein the solid substrate is a chip.

23. The method of claim 19, wherein the solid substrate is glass.

24. The method of claim 19, wherein the solid substrate is a multiwell plate.

25. The method of claim 19, wherein the solid substrate is a fiber optic cable.

26. The method of claim 1, wherein the probe polynucleotide is attached to a porous gel matrix.

27. The method of claim 1, wherein the sample is simultaneously contacted with a second probe polynucleotide under conditions in which the second probe polynucleotide can hybridize to a second amplification product, if present, from a second target polynucleotide, wherein the second probe polynucleotide comprises a second molecular beacon and a second quencher and a second fluorophore having different fluorescence characteristics than the first fluorophore, wherein a fluorescence emission from the second fluorophore is quenched by the second quencher under only one hybridization state of the second probe polynucleotide with the second target polynucleotide, and wherein a second light source is applied to the sample to excite the second fluorophore, and further determining whether the fluorescence emission from the second fluorophore is quenched or unquenched.

28. The method of claim 27, wherein the first and second probe polynucleotides are attached to a solid surface.

29. The method of claim 28, wherein a single quencher comprises both the first and second quenchers.

30. The method of claim 27, wherein the first and second quenchers comprise the same quenching material.

31. The method of claim 27, wherein a single light source comprises both the first and second light sources.

32. The method of claim 27, wherein the first and second amplification products are produced from first and second alleles of a single locus.

33. The method of claim 32, wherein the first and second alleles are two alleles of a single nucleotide polymorphism.

34. The method of claim 32, wherein one of the first and second alleles is an inactivating mutation of a tumor suppressor gene.

35. The method of claim 32, wherein one of the first and second alleles is an activating mutation of a cellular oncogene.

36. The method of claim 32, wherein one of the first and second alleles is an allele associated with an increased risk of a disease or disorder.

37. A method of forming an amplification product detection complex for assaying a sample for a first target polynucleotide, comprising:
providing a first primer and a second primer;
said first primer comprising a 3′ end, a first target complementary region that is complementary to the first target polynucleotide, said first target complementary region located at the 3′ end of the first primer, and a first target noncomplementary region that is not complementary to the first target polynucleotide at a position 3′ of a sequence to which the first target complementary region can hybridize;
said second primer comprising a 3′ end;
providing the sample, said sample suspected of containing the first target polynucleotide;
contacting the sample with the first primer under conditions in which the first target complementary region can hybridize to the first target polynucleotide and the first primer can be extended to form a first primer extension product;
altering the sample conditions to allow dissociation of the first primer extension product from the first target polynucleotide;
wherein the 3′ end of the second primer is complementary to the first primer extension product at a position in the first primer extension product that is 3′ to the first target complementary region;
contacting the sample with the second primer under conditions in which the second primer can hybridize to the first primer extension product and be extended to form a second primer extension product comprising a first capture sequence that is the complement of the first target noncomplementary region and does not exist elsewhere in the second primer extension product, wherein the second primer extension product is the amplification product;
altering the sample conditions to allow dissociation of the second primer extension product from the first primer extension product; and
contacting the sample with a probe polynucleotide comprising a molecular beacon comprising first and second complementary regions and a third region located between the first and second complementary regions, wherein the molecular beacon can form a stem-loop structure in which the first and second complementary regions hybridize to each other to form a stem and the third region forms a loop, wherein at least a part of the third region is complementary to at least a part of the capture sequence, such that the probe polynucleotide can preferentially hybridize to the second primer extension product and thereby disrupt formation of the stem-loop structure under at least one set of hybridization conditions, and wherein said contacting takes place under said set of conditions, to form an amplification product detection complex;
wherein the probe polynucleotide is linked to a first quencher and to a first fluorophore, wherein the first quencher and first fluorophore are located such that the first quencher can quench a fluorescence emission from the first fluorophore either under a first hybridization state when the probe polynucleotide is not hybridized to the second primer extension product and the stem-loop structure is formed or under a second hybridization state when the probe polynucleotide is hybridized to the second primer extension product and the stem-loop structure is not formed, but not under both hybridization states;
exciting the first fluorophore by applying a light source to the amplification product detection complex; and
determining the fluorescence emission from the first fluorophore.

38. The method of claim 37, wherein the target polynucleotide is DNA.

39. The method of claim 37, wherein the target polynucleotide is RNA.

40. The method of claim 39, wherein a polymerase having reverse transcriptase activity is used to form the first primer extension product from the target polynucleotide.

41. The method of claim 37, wherein the target polynucleotide is single-stranded.

42. The method of claim 37, wherein the target polynucleotide is double-stranded.

43. The method of claim 37, wherein the sample is again contacted with the first and second primers after altering the sample conditions to allow dissociation of the second primer extension product from the first primer extension product to form a plurality of first and second primer extension products.

44. The method of claim 37, wherein altering the sample conditions to allow dissociation of the first primer extension product from the target polynucleotide comprises raising the temperature of the sample.

45. The method of claim 37, wherein altering the sample conditions to allow dissociation of the second primer extension product from the first primer extension product comprises raising the temperature of the sample.

46. The method of claim 37, wherein the fluorescence emission from the first fluorophore is quenched under the first hybridization state.

47. The method of claim 37, wherein the fluorescence emission from the first fluorophore is quenched under the second hybridization state.

48. The method of claim 37, wherein the probe polynucleotide is present when the sample is contacted with the second primer.

49. The method of claim 37, wherein the fluorescence emission from the first fluorophore is monitored during formation of the second primer extension product.

50. The method of claim 37, wherein the fluorescence emission from the first fluorophore is monitored after formation of the second primer extension product.

51. The method of claim 37, further comprising removing single-stranded polynucleotides from the sample prior to altering the sample conditions to allow dissociation of the second primer extension product from the first primer extension product.

52. The method of claim 51, wherein removing single-stranded polynucleotides from the sample comprises adding a thermolabile single-stranded nuclease to the sample under conditions suitable and for a time sufficient to allow removal of single-stranded polynucleotides from the sample, and then heating the sample to inactivate the single-stranded nuclease.

53. The method of claim 37, wherein the target polynucleotide is a first allele of a locus having at least two alleles, and wherein the first primer preferentially is extended from the target polynucleotide as compared to another allele of the same locus.

54. The method of claim 53, wherein the first primer has at least one mismatch at one of the five 3′ nucleotides of the first primer with said another allele.

55. The method of claim 53, wherein the at least two alleles differ by a single polynucleotide.

56. The method of claim 37, further comprising concurrently assaying the same sample for a second target polynucleotide by employing, in the same steps:
a second pair of first and second primers to form corresponding first and second primer extension products from the second target polynucleotide, wherein the first primer of the second pair comprises a second target noncomplementary region, wherein the second primer extension product formed from the second target polynucleotide comprises a second capture sequence complementary to the second target noncomplementary region;
a second probe polynucleotide comprising a second molecular beacon wherein at least a part of the third region thereof is complementary to at least a part of the second capture sequence and further comprising a second fluorophore having detectably different emission characteristics from the first fluorophore and whose fluorescence emission is quenched in only one of the hybridization states of the second probe polynucleotide with the second primer extension product formed from the second target polynucleotide;
wherein the second probe polynucleotide can preferentially hybridize to the second primer extension product formed from the second target polynucleotide under said set of conditions;
and exciting the second fluorophore with the same or a different light source and determining if the fluorescence emission from the second fluorophore is quenched or unquenched.

57. The method of claim 56, wherein the first and second probe polynucleotides are linked and the first quencher can quench the fluorescence emissions from the first and second fluorophores independently based on the hybridization state of the first and second probe polynucleotides, respectively.

58. The method of claim 56, wherein the first and second probe polynucleotides are linked to a substrate that comprises the first quencher.

59. The method of claim 56, wherein the second probe polynucleotide comprises a second quencher, which may be the same as or different than the first quencher, wherein the second quencher quenches the fluorescence emission from the second fluorophore in only one of the hybridization states of the second probe polynucleotide.

60. The method of claim 56, wherein the first and second target polynucleotides are two alleles of the same locus.

61. The method of claim 60, wherein the two alleles differ by a single nucleotide.

62. An amplification product detection complex comprising a probe polynucleotide hybridized to an amplification product from a target polynucleotide, wherein the probe polynucleotide comprises a molecular beacon and the amplification product comprises a capture sequence not present in the target polynucleotide, wherein at least a part of a loop region of the molecular beacon is hybridized to at least a part of the capture sequence, and wherein the molecular beacon comprises a fluorophore whose fluorescence emission is different than when the probe polynucleotide is not hybridized to the amplification product.

63. A kit for assaying for an amplification product from a target polynucleotide comprising:
a probe polynucleotide that comprises a molecular beacon comprising a stem-loop structure, a quencher, and a fluorophore;
a first primer comprising a 3′ end, a first target complementary region that is complementary to the target polynucleotide, said first target complementary region located at the 3′ end of the first primer, and a first target noncomplementary region that is not complementary to the target polynucleotide at a position 3′ of a sequence to which the first target complementary region can hybridize;
a second primer that is complementary to a first primer extension product produced by extension of the first primer when hybridized to the target polynucleotide, said second primer able to form a second primer extension product that is the amplification product from the first primer extension product;
a housing for retaining the probe polynucleotide, first primer, and second primer; and
instructions provided with said housing that describe how to use the components of the kit to assay a sample by forming an amplification product from the target polynucleotide using the first and second primers that comprises a capture sequence that is complementary to the target noncomplementary region in the first primer, wherein said capture sequence can bind to the loop of the molecular beacon and disrupt the stem-loop structure.
